(19) United States
(12) Patent Application Publication
(10) Pub. No.: US 2023/0141887 A1
(43) Pub. Date: May 11, 2023

(54) CRYSTAL FORM OF DIAZASPIROPYRAN COMPOUND

(71) Applicant: MEDSHINE DISCOVERY INC., Nanjing, Jiangsu (CN)

(72) Inventors: Xiangyu FU, Shanghai (CN); Charles Z. DING, Shanghai (CN); Lihong HU, Shanghai (CN); Xu ZENG, Shanghai (CN); Ting YAO, Shanghai (CN)

(21) Appl. No.: 17/907,607

(22) PCT Filed: Mar. 30, 2021
(86) PCT No.: PCT/CN2021/084092

§ 371 Date: Sep. 28, 2022

(30) Foreign Application Priority Data
Mar. 30, 2020 (CN) 202010239276.0
Apr. 1, 2020 (CN) 202010251233.4

(51) Int. Cl. C07D 471/10

(52) U.S. Cl. CPC C07D 471/10; C07B 2200/13

(57) ABSTRACT

Disclosed are a salt type and a crystal form (I) of a diazaspiropyran compound, and a preparation method therefor. Also disclosed is the use of the salt type and the crystal form in the preparation of drugs for treating related diseases.

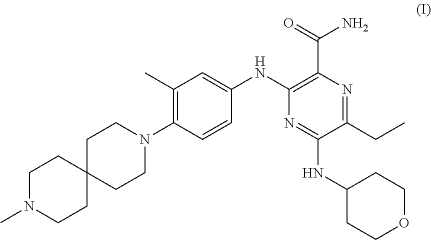 [SMILES: CCc1nc(C(N)=O)c(Nc2ccc(N3CCC4(CCN(C)CC4)CC3)c(C)c2)nc1NC1CCOCC1]

The present application is a United States National Phase under 35 U.S.C. § 371 of International Application No. PCT/CN2021/084092, filed Mar. 30, 2021, which claims the priority of: CN202010239276.0, filed on Mar. 30, 2020; and CN202010251233.4, filed on Apr. 1, 2020.

### FIELD OF THE INVENTION

The present disclosure relates to a salt form and a crystal form of a diazaspiro-pyran compound as well as a preparation method thereof, and also to the use of the salt form and the crystal form in the manufacture of a medicament for the treatment of related diseases.

### BACKGROUND OF THE INVENTION

Acute myeloid leukemia (AML) is the most common acute leukemia in adults and is a disease caused by the malignant proliferation of bone marrow hematopoietic cells. The incidence of AML is 3.4 per 100,000, and the median age of patients is 67 years. At present, the treatment of AML still needs to rely on chemotherapy, and about 70% of patients who have been relieved eventually relapse and become refractory leukemia. In addition, the prognosis of AML is poor, especially for elderly patients and patients with poor physical fitness. Drug resistance is the most important reason for the failure of treatment of AML, but the mechanism of drug resistance in leukemia is still unknown. Therefore, finding new targets and their inhibitors is of great significance for improving the therapeutic effect of AML and changing the prognosis.

The FLT3 receptor is a member of the type III receptor tyrosine kinase family FLT3 mutations are the most common genetic mutations in AML, mainly including internal tandem duplication (ITD) mutations in the juxtamembrane domain and point mutations (TKD) at the loop of FLT3. These mutations cause the downstream signaling pathway to be continuously activated, and the mutant cells toproliferate excessively. At present, FLT3 has been considered as an important target for the treatment of AML, and FLT3 inhibitors are also considered to be the most promising molecular targeted drugs for the treatment of AML.

AXL is also known as Ufo, Ark or Tyrol. Its abnormal expression can activate antagonism of tumor cell apoptosis, promote invasion and metastasis of tumor cells, and promote tumor angiogenesis, all of which drive the occurrence and development of tumors. High expression of AXL will cause reduced survival and worse prognosis for patients with AML. In addition, the overexpression of AXL is closely related to the drug resistance of targeted drugs and chemotherapeutic drugs. Recently, AXL has also been found to have potential in immunotherapy. Therefore, the development of dual inhibitors of FLT3 and AXL is expected to achieve better efficacy in the treatment of AML.

WO2012053606A1 reports Compound A (Example 176 in WO2012053606A1), mentioning that such type of molecules has FLT3 inhibitory activity and can be used for the treatment of AML, but no specific test data is given therein.

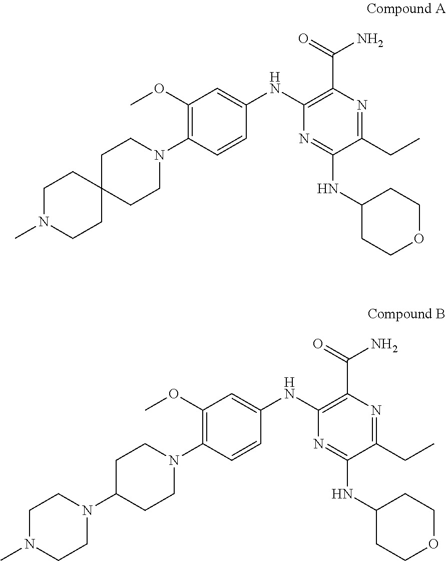

WO2010128659A1 reports Compound B with FLT3 inhibitory activity (Example 547 in WO2010128659A1). Phase III clinical trials of this compound for the treatment of relapsed or refractory AML are underway.

### SUMMARY OF THE INVENTION

The present disclosure provides a crystal form A of the compound of formula (I), which is characterized by X-ray powder diffraction pattern with characteristic diffraction peaks at 2θ angles of 15.48±0.20°, 19.32±0.20°, and 20.17±0.20°.

In some embodiments of the present disclosure, disclosed is the above crystal form A of the compound of formula (I), which is characterized by X-ray powder diffraction pattern with characteristic diffraction peaks at 2θ angles of 8.26±0.20°, 14.06±0.20°, 14.83±0.20°, 15.48±0.20°, 18.60±0.20°, 19.32±0.20°, 20.17±0.20°, and 24.28±0.20°.

In some embodiments of the present disclosure, disclosed is the above crystal form A of the compound of formula (I), which is characterized by X-ray powder diffraction pattern with characteristic diffraction peaks at 2θ angles of 8.26±0.20°, 12.36±0.20°, 14.06±0.20°, 14.83±0.20°, 15.48±0.20°, 16.55±0.20°, 17.29±0.20°, 18.60±0.20°, 19.32±0.20°, 20.17±0.20°, 24.28±0.20°, and 25.51±0.20°.

In some embodiments of the present disclosure, disclosed is the above crystal form A of the compound of formula (I), which is characterized by X-ray powder diffraction pattern with characteristic diffraction peaks at 2θ angles of 8.26°, 9.13°, 11.47°, 12.36°, 13.37°, 14.06°, 14.83°, 15.48°, 16.55°, 17.29°, 17.90°, 18.60°, 18.99°, 19.32°, 20.17°, 20.49°, 22.00°, 24.28°, 24.83°, 25.51°, 28.11°, and 30.70°. In some embodiments of the present disclosure, disclosed is the above crystal form A of the compound of formula (I), which has a XRPD pattern as shown in FIG. 1.

In some embodiments of the present disclosure, the XRPD pattern resolution data of the above crystal form A is shown in Table 1:

TABLE 1
XRPD pattern resolution data of the crystal form A

| | | | Relative |
|---|---|---|---|
| | 2θ | D-spacing | intensity |
| No. | Angle[°] | [Å] | [%] |
| 1 | 8.26 | 10.70 | 44.45 |
| 2 | 9.13 | 9.68 | 4.62 |
| 3 | 11.47 | 7.71 | 5.41 |
| 4 | 12.36 | 7.16 | 15.36 |
| 5 | 13.37 | 6.62 | 9.59 |
| 6 | 14.06 | 6.30 | 25.07 |
| 7 | 14.83 | 5.97 | 17.73 |
| 8 | 15.48 | 5.73 | 100.00 |
| 9 | 16.55 | 5.36 | 13.09 |
| 10 | 17.29 | 5.13 | 11.60 |
| 11 | 17.90 | 4.96 | 9.87 |
| 12 | 18.60 | 4.77 | 30.21 |
| 13 | 18.99 | 4.67 | 35.90 |
| 14 | 19.32 | 4.60 | 78.39 |
| 15 | 20.17 | 4.40 | 52.48 |
| 16 | 20.49 | 4.33 | 42.99 |
| 17 | 22.00 | 4.04 | 11.53 |
| 18 | 24.28 | 3.67 | 31.13 |
| 19 | 24.83 | 3.59 | 8.21 |
| 20 | 25.51 | 3.49 | 11.92 |
| 21 | 28.11 | 3.17 | 5.91 |
| 22 | 30.70 | 2.91 | 1.78 |

In some embodiments of the present disclosure, disclosed is the above crystal form A of the compound of formula (I), which has a thermogravimetric analysis curve with a weight loss of up to 2.65% at 150.0±3° C.

In some embodiments of the present disclosure, disclosed is the above crystal form A of the compound of formula (I), which has a TGA curve as shown in FIG. 2.

In some embodiments of the present disclosure, disclosed is the above crystal form A of the compound of formula (I), which has a differential scanning calorimetry curve with a starting point of the endothermic peak at 237.1±5° C.

In some embodiments of the present disclosure, disclosed is the above crystal form A of the compound of formula (I), which has a DSC curve as shown in FIG. 3.

The present disclosure also provides a crystal form B of the compound of formula (I), which is characterized by X-ray powder diffraction pattern with characteristic diffraction peaks at 2θ angles of 14.11±0.20°, 19.29±0.20°, and 21.22±0.20°.

In some embodiments of the present disclosure, disclosed is the above crystal form B of the compound of formula (I), which is characterized by X-ray powder diffraction pattern with characteristic diffraction peaks at 2θ angles of 7.57±0.20°, 14.11±0.20°, 15.16±0.20°, 18.74±0.20°, 19.29±0.20°, 20.68±0.20°, 21.22±0.20°, and 24.28±0.20°.

In some embodiments of the present disclosure, disclosed is the above crystal form B of the compound of formula (I), which is characterized by X-ray powder diffraction pattern with characteristic diffraction peaks at 2θ angles of 7.05±0.20°, 7.57±0.20°, 14.11±0.20°, 15.16±0.20°, 15.68±0.20°, 17.69±0.20°, 18.74±0.20°, 19.29±0.20°, 20.68±0.20°, 21.22±0.20°, 24.28±0.20°, and 25.17±0.20°.

In some embodiments of the present disclosure, disclosed is the above crystal form B of the compound of formula (I), which is characterized by X-ray powder diffraction pattern with characteristic diffraction peaks at 2θ angles of 7.05°, 7.57°, 7.97°, 9.29°, 10.48°, 13.48°, 14.11°, 15.16°, 15.68°, 17.69°, 18.74°, 19.29°, 20.12°, 20.68°, 21.22°, 24.28°, 25.17°, 27.86°, 30.43°, and 31.50°. In some embodiments of the present disclosure, disclosed is the above crystal form B of the compound of formula (I), which has a XRPD pattern as shown in FIG. 5.

In some embodiments of the present disclosure, the XRPD pattern resolution data of the above crystal form B is shown in Table 2:

TABLE 2
XRPD pattern resolution data of the crystal form B

| | | | Relative |
|---|---|---|---|
| | 2θ | D-spacing | intensity |
| No. | Angle[°] | [Å] | [%] |
| 1 | 7.05 | 12.53 | 54.62 |
| 2 | 7.57 | 11.68 | 55.16 |
| 3 | 7.97 | 11.09 | 19.87 |
| 4 | 9.29 | 9.52 | 15.11 |
| 5 | 10.48 | 8.44 | 22.54 |
| 6 | 13.48 | 6.57 | 11.25 |
| 7 | 14.11 | 6.28 | 81.85 |
| 8 | 15.16 | 5.85 | 56.65 |
| 9 | 15.68 | 5.65 | 47.27 |
| 10 | 17.69 | 5.02 | 39.47 |
| 11 | 18.74 | 4.73 | 63.91 |
| 12 | 19.29 | 4.60 | 100.00 |
| 13 | 20.12 | 4.41 | 17.13 |
| 14 | 20.68 | 4.30 | 66.74 |
| 15 | 21.22 | 4.19 | 94.43 |
| 16 | 24.28 | 3.67 | 81.01 |
| 17 | 25.17 | 3.54 | 33.41 |
| 18 | 27.86 | 3.20 | 18.36 |
| 19 | 30.43 | 2.94 | 6.83 |
| 20 | 31.50 | 2.84 | 6.39 |

In some embodiments of the present disclosure, disclosed is the above crystal form B of the compound of formula (I), which has a thermogravimetric analysis curve with a weight loss of up to 4.20% at 140.0±3° C.

In some embodiments of the present disclosure, disclosed is the above crystal form B of the compound of formula (I), which has a TGA curve as shown in FIG. 6.

In some embodiments of the present disclosure, disclosed is the above crystal form B of the compound of formula (I), which has a differential scanning calorimetry curve with a starting point of the endothermic peak at 237.2±5° C.

In some embodiments of the present disclosure, disclosed is the above crystal form B of the compound of formula (I), which has a DSC curve as shown in FIG. 7.

The present disclosure also provides a crystal form C of the compound of formula (I), which is characterized by X-ray powder diffraction pattern with characteristic diffraction peaks at 2θ angles of 8.26±0.20°, 19.30±0.20°, and 20.53±0.20°.

In some embodiments of the present disclosure, disclosed is the above crystal form C of the compound of formula (I), which is characterized by X-ray powder diffraction pattern with characteristic diffraction peaks at 2θ angles of 8.26±0.20°, 12.36±0.20°, 14.07±0.20°, 15.45±0.20°, 18.59±0.20°, 19.30±0.20°, 20.53±0.20°, and 24.29±0.20°.

In some embodiments of the present disclosure, disclosed is the above crystal form C of the compound of formula (I), which is characterized by X-ray powder diffraction pattern with characteristic diffraction peaks at 2θ angles of 8.26±0.20°, 12.36±0.20°, 14.07±0.20°, 15.45±0.20°, 16.54±0.20°, 17.32±0.20°, 18.59±0.20°, 19.30±0.20°, 20.53±0.20°, 24.29±0.20°, 24.89±0.20°, and 25.49±0.20°.

In some embodiments of the present disclosure, disclosed is the above crystal form C of the compound of formula (I), which is characterized by X-ray powder diffraction pattern with characteristic diffraction peaks at 2θ angles of 5.59°, 8.26°, 9.27°, 12.36°, 13.63°, 14.07°, 14.81°, 15.45°, 16.54°, 17.32°, 18.59°, 18.95°, 19.30°, 20.13°, 20.53°, 21.27°, 21.80°, 24.29°, 24.89°, 25.49°, 27.35°, 28.10°, 28.59°, 29.32°, 30.33°, 30.83°, 32.16°, 33.46°, and 36.60°. In some embodiments of the present disclosure, disclosed is the above crystal form C of the compound of formula (I), which has a XRPD pattern as shown in FIG. 8.

In some embodiments of the present disclosure, the XRPD pattern resolution data of the above crystal form C is shown in Table 3:

TABLE 3
XRPD pattern resolution data of the crystal form C

| | | | Relative |
|---|---|---|---|
| | 2θ | D-spacing | intensity |
| No. | Angle[°] | [Å] | [%] |
| 1 | 5.59 | 15.81 | 3.96 |
| 2 | 8.26 | 10.70 | 100.00 |
| 3 | 9.27 | 9.54 | 7.62 |
| 4 | 12.36 | 7.16 | 37.79 |
| 5 | 13.63 | 6.50 | 6.77 |
| 6 | 14.07 | 6.30 | 43.44 |
| 7 | 14.81 | 5.98 | 7.49 |
| 8 | 15.45 | 5.73 | 37.22 |
| 9 | 16.54 | 5.36 | 10.48 |
| 10 | 17.32 | 5.12 | 19.86 |
| 11 | 18.59 | 4.77 | 34.60 |
| 12 | 18.95 | 4.68 | 13.40 |
| 13 | 19.30 | 4.60 | 45.47 |
| 14 | 20.13 | 4.41 | 25.36 |
| 15 | 20.53 | 4.33 | 81.22 |
| 16 | 21.27 | 4.18 | 4.67 |
| 17 | 21.80 | 4.08 | 2.25 |
| 18 | 24.29 | 3.66 | 24.40 |
| 19 | 24.89 | 3.58 | 16.09 |
| 20 | 25.49 | 3.49 | 9.90 |
| 21 | 27.35 | 3.26 | 4.90 |
| 22 | 28.10 | 3.18 | 4.19 |
| 23 | 28.59 | 3.12 | 8.22 |
| 24 | 29.32 | 3.05 | 1.82 |
| 25 | 30.33 | 2.95 | 2.16 |
| 26 | 30.83 | 2.90 | 3.31 |
| 27 | 32.16 | 2.78 | 3.52 |
| 28 | 33.46 | 2.68 | 1.88 |
| 29 | 36.60 | 2.45 | 1.64 |

In some embodiments of the present disclosure, disclosed is the above crystal form C of the compound of formula (I), which has a thermogravimetric analysis curve with a weight loss of up to 0.71% at 220.0±3° C.

In some embodiments of the present disclosure, disclosed is the above crystal form C of the compound of formula (I), which has a TGA curve as shown in FIG. 9.

In some embodiments of the present disclosure, disclosed is the above crystal form C of the compound of formula (I), which has a differential scanning calorimetry curve with a starting point of the endothermic peak at 238.1±5° C.

In some embodiments of the present disclosure, disclosed is the above crystal form C of the compound of formula (I), which has a DSC curve as shown in FIG. 10.

The present disclosure also provides a crystal form D of the compound of formula (I), which is characterized by X-ray powder diffraction pattern with characteristic diffraction peaks at 2θ angles of 7.97±0.20°, 15.47±0.20°, and 19.01±0.20°.

In some embodiments of the present disclosure, disclosed is the above crystal form D of the compound of formula (I), which is characterized by X-ray powder diffraction pattern with characteristic diffraction peaks at 2θ angles of 6.76±0.20°, 7.97±0.20°, 13.52±0.20°, 14.00±0.20°, 15.47±0.20°, 19.01±0.20°, 19.51±0.20°, and 20.40±0.20°.

In some embodiments of the present disclosure, disclosed is the above crystal form D of the compound of formula (I), which is characterized by X-ray powder diffraction pattern with characteristic diffraction peaks at 2θ angles of 4.98±0.20°, 6.76±0.20°, 7.97±0.20°, 13.52±0.20°, 14.00±0.20°, 15.47±0.20°, 16.01±0.20°, 18.34±0.20°, 19.01±0.20°, 19.51±0.20°, 20.40±0.20°, and 20.85±0.20°.

In some embodiments of the present disclosure, disclosed is the above crystal form D of the compound of formula (I), which is characterized by X-ray powder diffraction pattern with characteristic diffraction peaks at 2θ angles of 4.98°, 6.76°, 7.97°, 9.50°, 11.45°, 11.96°, 13.52°, 14.00°, 15.47°, 16.01°, 16.51°, 16.96°, 17.75°, 18.34°, 19.01°, 19.51°, 20.40°, 20.85°, 23.28°, 26.47°, and 28.60°.

In some embodiments of the present disclosure, disclosed is the above crystal form D of the compound of formula (I), which has a XRPD pattern as shown in FIG. 11.

In some embodiments of the present disclosure, the XRPD pattern resolution data of the above crystal form D is shown in Table 4:

TABLE 4
XRPD pattern resolution data of the crystal form D

| | | | Relative |
|---|---|---|---|
| | 2θ | D-spacing | intensity |
| No. | Angle[°] | [Å] | [%] |
| 1 | 4.98 | 17.76 | 26.61 |
| 2 | 6.76 | 13.09 | 40.84 |
| 3 | 7.97 | 11.09 | 100.00 |
| 4 | 9.50 | 9.31 | 18.48 |
| 5 | 11.45 | 7.73 | 16.75 |
| 6 | 11.96 | 7.40 | 11.76 |
| 7 | 13.52 | 6.55 | 40.41 |
| 8 | 14.00 | 6.33 | 40.76 |
| 9 | 15.47 | 5.73 | 45.40 |
| 10 | 16.01 | 5.54 | 35.34 |
| 11 | 16.51 | 5.37 | 18.62 |
| 12 | 16.96 | 5.23 | 16.86 |
| 13 | 17.75 | 5.00 | 12.72 |
| 14 | 18.34 | 4.84 | 22.99 |
| 15 | 19.01 | 4.67 | 67.26 |
| 16 | 19.51 | 4.55 | 37.62 |
| 17 | 20.40 | 4.35 | 41.46 |
| 18 | 20.85 | 4.26 | 36.57 |
| 19 | 23.28 | 3.82 | 22.72 |
| 20 | 26.47 | 3.37 | 3.80 |
| 21 | 28.60 | 3.12 | 4.90 |

In some embodiments of the present disclosure, disclosed is the above crystal form D of the compound of formula (I), which has a thermogravimetric analysis curve with a weight loss of up to 1.06% at 220.0±3° C.

In some embodiments of the present disclosure, disclosed is the above crystal form D of the compound of formula (I), which has a TGA curve as shown in FIG. 12.

In some embodiments of the present disclosure, disclosed is the above crystal form D of the compound of formula (I), which has a differential scanning calorimetry curve with a starting point of the endothermic peak at 237.0±5° C.

In some embodiments of the present disclosure, disclosed is the above crystal form D of the compound of formula (I), which has a DSC curve as shown in FIG. 13.

The present disclosure also provides a crystal form E of the compound of formula (I), which is characterized by X-ray powder diffraction pattern with characteristic diffraction peaks at 2θ angles of 8.10±0.20°, 9.92±0.20°, and 21.91±0.20°.

In some embodiments of the present disclosure, disclosed is the above crystal form E of the compound of formula (I), which is characterized by X-ray powder diffraction pattern with characteristic diffraction peaks at 2θ angles of 6.97±0.20°, 8.10±0.20°, 9.92±0.20°, 15.28±0.20°, 16.72±0.20°, 18.02±0.20°, 20.00±0.20°, and 21.91±0.20°.

In some embodiments of the present disclosure, disclosed is the above crystal form E of the compound of formula (I), which is characterized by X-ray powder diffraction pattern with characteristic diffraction peaks at 2θ angles of 6.97±0.20°, 8.10±0.20°, 9.92±0.20°, 10.55±0.20°, 11.35±0.20°, 15.28±0.20°, 15.89±0.20°, 16.72±0.20°, 18.02±0.20°, 20.00±0.20°, 21.91±0.20°, and 22.56±0.20°.

In some embodiments of the present disclosure, disclosed is the above crystal form E of the compound of formula (I), which is characterized by X-ray powder diffraction pattern with characteristic diffraction peaks at 2θ angles of 6.97°, 8.10°, 9.92°, 10.55°, 10.92°, 11.35°, 11.65°, 12.88°, 13.91°, 15.28°, 15.89°, 16.38°, 16.72°, 17.05°, 17.56°, 18.02°, 18.31°, 20.00°, 21.15°, 21.91°, 22.56°, 22.87°, 23.40°, 23.90°, 24.66°, 25.43°, 25.75°, 26.44°, 27.91°, 28.84°, 29.26°, 32.17°, 33.03°, 34.25°, 36.22°, 37.07°, and 38.45°.

In some embodiments of the present disclosure, disclosed is the above crystal form E of the compound of formula (I), which has a XRPD pattern as shown in FIG. 14.

In some embodiments of the present disclosure, the XRPD pattern resolution data of the above crystal form E is shown in Table 5:

TABLE 5
XRPD pattern resolution data of the crystal form E

| | | | Relative |
|---|---|---|---|
| | 2θ | D-spacing | intensity |
| No. | Angle[°] | [Å] | [%] |
| 1 | 6.97 | 12.69 | 43.32 |
| 2 | 8.10 | 10.92 | 68.29 |
| 3 | 9.92 | 8.91 | 53.60 |
| 4 | 10.55 | 8.38 | 14.42 |
| 5 | 10.92 | 8.10 | 8.58 |
| 6 | 11.35 | 7.80 | 12.32 |
| 7 | 11.65 | 7.60 | 9.15 |
| 8 | 12.88 | 6.87 | 2.63 |
| 9 | 13.91 | 6.37 | 2.52 |
| 10 | 15.28 | 5.80 | 29.69 |
| 11 | 15.89 | 5.58 | 15.41 |
| 12 | 16.38 | 5.41 | 17.93 |
| 13 | 16.72 | 5.30 | 38.51 |
| 14 | 17.05 | 5.20 | 12.22 |
| 15 | 17.56 | 5.05 | 6.65 |
| 16 | 18.02 | 4.92 | 17.64 |
| 17 | 18.31 | 4.85 | 9.08 |
| 18 | 20.00 | 4.44 | 26.93 |
| 19 | 21.15 | 4.20 | 11.76 |
| 20 | 21.91 | 4.06 | 100.00 |
| 21 | 22.56 | 3.94 | 13.23 |
| 22 | 22.87 | 3.89 | 6.83 |
| 23 | 23.40 | 3.80 | 11.07 |
| 24 | 23.90 | 3.72 | 2.75 |
| 25 | 24.66 | 3.61 | 5.49 |
| 26 | 25.43 | 3.50 | 9.18 |
| 27 | 25.75 | 3.46 | 11.26 |
| 28 | 26.44 | 3.37 | 4.52 |
| 29 | 27.91 | 3.20 | 3.93 |
| 30 | 28.84 | 3.10 | 10.48 |
| 31 | 29.26 | 3.05 | 3.27 |
| 32 | 32.17 | 2.78 | 7.14 |
| 33 | 33.03 | 2.71 | 2.02 |
| 34 | 34.25 | 2.62 | 0.85 |
| 35 | 36.22 | 2.48 | 0.96 |
| 36 | 37.07 | 2.43 | 1.47 |
| 37 | 38.45 | 2.34 | 0.93 |

In some embodiments of the present disclosure, disclosed is the above crystal form E of the compound of formula (I), which has a thermogravimetric analysis curve with a weight loss of up to 9.42% at 150.0±3° C.

In some embodiments of the present disclosure, disclosed is the above crystal form E of the compound of formula (I), which has a TGA curve as shown in FIG. 15.

In some embodiments of the present disclosure, disclosed is the above crystal form E of the compound of formula (I), which has a differential scanning calorimetry curve with starting points of the endothermic peaks at 123.1±5° C. and 237.0±5° C.

In some embodiments of the present disclosure, disclosed is the above crystal form E of the compound of formula (I), which has a DSC curve as shown in FIG. 16.

The present disclosure also provides a crystal form F of the compound of formula (I), which is characterized by X-ray powder diffraction pattern with characteristic diffraction peaks at 2θ angles of 8.30±0.20°, 15.49±0.20°, and 19.31±0.20°.

In some embodiments of the present disclosure, disclosed is the above crystal form F of the compound of formula (I), which is characterized by X-ray powder diffraction pattern with characteristic diffraction peaks at 2θ angles of 8.30±0.20°, 12.40±0.20°, 15.49±0.20°, 17.36±0.20°, 18.60±0.20°, 19.31±0.20°, 20.14±0.20°, and 20.55±0.20°.

In some embodiments of the present disclosure, disclosed is the above crystal form F of the compound of formula (I), which is characterized by X-ray powder diffraction pattern with characteristic diffraction peaks at 2θ angles of 8.30±0.20°, 12.40±0.20°, 14.10±0.20°, 15.49±0.20°, 16.57±0.20°, 17.36±0.20°, 18.60±0.20°, 19.31±0.20°, 20.14±0.20°, 20.55±0.20°, 24.28±0.20°, and 24.91±0.20°.

In some embodiments of the present disclosure, disclosed is the above crystal form F of the compound of formula (I), which is characterized by X-ray powder diffraction pattern with characteristic diffraction peaks at 2θ angles of 5.60°, 6.86°, 8.30°, 9.30°, 12.40°, 13.35°, 13.69°, 14.10°, 14.84°, 15.49°, 16.01°, 16.57°, 16.78°, 17.36°, 18.01°, 18.60°, 18.97°, 19.31°, 20.14°, 20.55°, 21.22°, 21.78°, 23.93°, 24.28°, 24.91°, 25.50°, 26.24°, 27.58°, 28.20°, 28.62°, 29.71°, 30.33°, 30.83°, 32.16°, 33.67°, 35.19°, 36.47°, and 37.71°.

In some embodiments of the present disclosure, disclosed is the above crystal form F of the compound of formula (I), which has a XRPD pattern as shown in FIG. 17.

In some embodiments of the present disclosure, the XRPD pattern resolution data of the above crystal form F is shown in Table 6:

TABLE 6
XRPD pattern resolution data of the crystal form F

| | | | Relative |
|---|---|---|---|
| | 2θ | D-spacing | intensity |
| No. | Angle[°] | [Å] | [%] |
| 1 | 5.60 | 15.78 | 7.36 |
| 2 | 6.86 | 12.88 | 2.87 |
| 3 | 8.30 | 10.66 | 76.32 |
| 4 | 9.30 | 9.51 | 6.92 |
| 5 | 12.40 | 7.14 | 58.20 |
| 6 | 13.35 | 6.63 | 7.65 |
| 7 | 13.69 | 6.47 | 8.06 |
| 8 | 14.10 | 6.28 | 28.69 |
| 9 | 14.84 | 5.97 | 17.34 |
| 10 | 15.49 | 5.72 | 100.00 |
| 11 | 16.01 | 5.54 | 7.51 |
| 12 | 16.57 | 5.35 | 18.51 |
| 13 | 16.78 | 5.28 | 15.42 |
| 14 | 17.36 | 5.11 | 51.62 |
| 15 | 18.01 | 4.92 | 4.20 |
| 16 | 18.60 | 4.77 | 30.82 |
| 17 | 18.97 | 4.68 | 35.03 |
| 18 | 19.31 | 4.60 | 62.94 |
| 19 | 20.14 | 4.41 | 34.26 |
| 20 | 20.55 | 4.32 | 49.48 |
| 21 | 21.22 | 4.19 | 7.51 |
| 22 | 21.78 | 4.08 | 4.27 |
| 23 | 23.93 | 3.72 | 6.93 |
| 24 | 24.28 | 3.67 | 24.36 |
| 25 | 24.91 | 3.58 | 26.30 |
| 26 | 25.50 | 3.49 | 10.50 |
| 27 | 26.24 | 3.40 | 3.97 |
| 28 | 27.58 | 3.23 | 4.88 |
| 29 | 28.20 | 3.16 | 9.96 |
| 30 | 28.62 | 3.12 | 6.67 |
| 31 | 29.71 | 3.01 | 3.10 |
| 32 | 30.33 | 2.95 | 2.47 |
| 33 | 30.83 | 2.90 | 2.44 |
| 34 | 32.16 | 2.78 | 3.98 |
| 35 | 33.67 | 2.66 | 1.00 |
| 36 | 35.19 | 2.55 | 2.46 |
| 37 | 36.47 | 2.46 | 2.58 |
| 38 | 37.71 | 2.39 | 1.90 |

In some embodiments of the present disclosure, disclosed is the above crystal form F of the compound of formula (I), which has a thermogravimetric analysis curve with a weight loss of up to 1.40% at 200.0±3° C.

In some embodiments of the present disclosure, disclosed is the above crystal form F of the compound of formula (I), which has a TGA curve as shown in FIG. 18.

In some embodiments of the present disclosure, disclosed is the above crystal form F of the compound of formula (I), which has a differential scanning calorimetry curve with a starting point of the endothermic peak at 236.4±5° C.

In some embodiments of the present disclosure, disclosed is the above crystal form F of the compound of formula (I), which has a DSC curve as shown in FIG. 19.

The present disclosure also provides use of the above crystal form A, crystal form B, crystal form C, crystal form D, crystal form E, or crystal form F in the manufacture of a medicament for the treatment of diseases associated with FLT3 and/or AXL.

In some embodiments of the present disclosure, disclosed is the above use, wherein the disease is AML.

### Technical Effect

The present disclosure provides a novel FLT3/AXL dual inhibitor, and a crystal form and a salt form thereof. Compared with the prior inhibitors, the inhibitor has unexpectedly higher in vitro enzyme activity and cell activity. Especially, the inhibitor has significant advantages in the enzyme activity test for FLT3 mutation. The pharmacokinetic properties of the inhibitor are better than that of the prior inhibitors. In the in vivo assay for MV4-11, a low dose of the compound of the present disclosure shows a good tumor inhibitory activity. The drug withdrawal-rebound assay (MV4-11 assay) proves that the compound of the present disclosure has strong sustained tumor inhibitory ability. In the in vivo assay for Molm-13, the compound of the present disclosure shows unexpectedly excellent tumor inhibitory effect, which is obviously better than that of the prior compounds. The crystal forms provided herein have ideal solubility and good stability.

### Definition and Description

Unless otherwise specified, the following terms and phrases used herein are intended to have the following meanings. A specific term or phrase should not be considered indefinite or unclear in the absence of a particular definition, but should be understood in the conventional sense. When a trade name appears herein, it is intended to refer to its corresponding commodity or active ingredient thereof.

The intermediate compounds of the present disclosure can be prepared by a variety of synthetic methods well known to those skilled in the art, including the specific embodiments listed below, the embodiments formed by combining the specific embodiments listed below with other chemical synthetic methods, and the equivalent alternative methods well known to those skilled in the art. The alternative embodiments include, but are not limited to, the examples of the present disclosure.

The chemical reactions in the specific embodiments of the present disclosure are completed in a suitable solvent, which must be suitable for the chemical changes of the present disclosure and the reagents and materials required. In order to obtain the compound of the present disclosure, it is sometimes necessary for those skilled in the art to modify or select synthetic steps or reaction schemes based on the existing embodiments.

The present disclosure will be described in detail below through examples, which are not intended to limit the present disclosure.

All solvents used in the present disclosure are commercially available and can be used without further purification.

The following abbreviations are used in the present disclosure: MW represents microwave; r.t. represents room temperature; aq represents aqueous solution; DCM represents dichloromethane; THF represents tetrahydrofuran; DMSO represents dimethylsulfoxide; NMP represents N-methylpyrrolidone; EtOAc represents ethyl acetate; EtOH represents ethanol; MeOH represents methanol; dioxane represents dioxacyclohexane; HOAc represents acetic acid; Boc represents tert-butoxycarbonyl and Cbz represents benzyloxycarbonyl, both of which are amine protecting groups; Boc₂O represents di-tert-butyl dicarbonate; DIPEA represents diisopropylethylamine; TEA or Et₃N represents triethylamine; BnNH₂ represents benzylamine; PMBNH₂ represents p-methoxybenzylamine; KOAc represents potassium acetate; NaOAc represents sodium acetate; Cs₂CO₃ represents cesium carbonate; K₂CO₃ represents potassium carbonate; NaHCO₃ represents sodium bicarbonate; Na₂SO₄ represents sodium sulfate; Pyr represents pyridine; NaOH represents sodium hydroxide; TEA or Et₃N represents triethylamine; NaH represents sodium hydride; LiHMDS represents lithium bis(trimethylsilyl)amide; i-PrMgBr represents isopropylmagnesium bromide; t-BuOK represents potassium tert-butoxide; t-BuONa represents sodium tert-butoxide; Pd₂(dba)₃ represents tris(dibenzylideneacetone)dipalladium; Pd(PPh₃)₄ represents tetrakis(triphenylphosphine)palladium; Pd(dppf)Cl₂CH₂Cl₂ represents [1,1′-bis(diphenylphosphino)ferrocene[dichloropalladium.dichloromethane; Pd(OAc)₂ represents palladium acetate; Pd(PPh₃)₂Cl₂ represents di(triphenylphosphine)palladium dichloride; Pd(PPh₃)₃Cl represents tris(triphenylphosphine)rhodium chloride; Pd(OH)₂ represents palladium hydroxide; Xantphos represents 4,5-bis(diphenylphosphino)-9,9-dimethylxanthene; Xphos represents 2-dicyclohexylphosphino-2′,4′,6′-triisopropylbiphenyl; BINAP represents (±)-2,2′-bis-(diphenylphosphino)-1,1′-binaphthyl; Xantphos represents 4,5-bis-(diphenylphosphino)-9,9-dimethylxanthene; Xphos-Pd-G1 represents chloro(2-dicyclohexylphosphino-2′,4′,6′-triisopropyl-1,1′-biphenyl) [2-(2′-aminoethyl phenyl)]palladium(II); Xphos-PD-G₂ represents chloro(2-dicyclohexylphosphino-2′,4′,6′-triisopropyl-1,1′-biphenyl) [2-(2′-amino-1,1′-biphenyl)]palladium(II); Xphos-Pd-G3 represents methanesulfonato(2-dicyclohexylphosphino-2′,4′,6′-tri-isopropyl-1,1′-biphenyl) [2-(2′-amino-1,1′-biphenyl)]palladium(II); I₂ represents iodine; LiCl represents lithium chloride; HCl represents hydrochloric acid; and maleic acid represents cis-2-butenedioic acid.

The structures of compounds of the present disclosure can be confirmed by conventional methods well known to those skilled in the art. If the present disclosure relates to an absolute configuration of a compound, the absolute configuration can be confirmed by conventional techniques in the art, such as single crystal X-Ray diffraction (SXRD). In the single crystal X-Ray diffraction (SXRD), the diffraction intensity data of the cultivated single crystal is collected using a Bruker D8 venture diffractometer with a light source of CuKα radiation in a scanning mode of φ/ω scan; after collecting the relevant data, the crystal structure is further analyzed by the direct method (Shelxs97) to confirm the absolute configuration.

### X-Ray Powder Diffraction (XRPD) Method Used in the Present Disclosure

Instrument model: X'Pert³ X-ray diffractometer from PANalytical

Test method: About 10 mg of sample was used for XRPD detection.

Detailed XRPD parameters were as follows:

Radiation source: Cu, kα (Kα1=1.540598 Å, Kα2=1.544426 Å, Kα2/Kα1 intensity ratio: 0.5)
Voltage of a light tube: 45 kV, current of a light tube: 40 mA
Divergence slit: Fixed ⅛ deg
First soller slit: 0.04 rad, second soller slit: 0.04 rad
Receiving slit: none, anti-scatter slit: 7.5 mm
Measuring time: 5 min
Range of scanning angle: 3-40 deg
Step-width angle: 0.0263 deg
Step length: 46.665 second
Rotation speed of a sample disk: 15 rpm

### Differential Scanning Calorimetry (DSC) Method Used in the Present Disclosure

Instrument model: TA 2500 Differential Scanning calorimeter

Test method: Sample (about 1.5 mg) was placed in a DSC aluminum pot for testing, and the gland of the aluminum pot was not punctured. Under the condition of 50 mL/min N₂, the sample was heated from 25° C. (room temperature) to just before sample decomposition at a heating rate of 10° C./min.

### Thermogravimetric Analysis (TGA) Method Used in the Present Disclosure

Instrument model: TA 5500/Q5000 Thermogravimeter

Test method: Sample (about 1.5 mg) was placed in an open TGA aluminum pot for testing. Under the condition of 10 to 25 mL/min N₂, the sample was heated from room temperature to 350° C. at a heating rate of 10° C./min

### Dynamic Vapor Sorption (DVS) Method Used in the Present Disclosure

Instrument model: Intrinsic dynamic vapor sorption apparatus

Test condition: Sample (10 to 30 mg) was placed in a DVS sample pan for testing.

Detailed DVS parameters were as follows:

### Temperature: 25° C.

Equilibration: dm/dt=0.002%/min (The shortest time: 10 min, and the longest time: 180 min)
RH (%) test gradient: 10 (0-90%), 5 (90-95%)
Range of RH (%) test gradient: 0-95-0%

Tablet Test Method

Tablet-pressing equipment: SYP-5BS from Shanghai Xinnuo Instruments Co.

Tablet-pressing method: Sample powder was added to a circular mold (6 mm diameter) and pressed until the pressure reached about 350 MPa.

### BRIEF DESCRIPTION OF THE DRAWINGS

FIG. 1 is the XRPD pattern of the crystal form A of the compound of formula (I).

FIG. 2 is the TGA curve of the crystal form A of the compound of formula (I).

FIG. 3 is the DSC curve of the crystal form A of the compound of formula (I).

FIG. 4 is the DVS pattern of the crystal form A of the compound of formula (I).

FIG. 5 is the XRPD pattern of the crystal form B of the compound of formula (I).

FIG. 6 is the TGA curve of the crystal form B of the compound of formula (I).

FIG. 7 is the DSC curve of the crystal form B of the compound of formula (I).

FIG. 8 is the XRPD pattern of the crystal form C of the compound of formula (I).

FIG. 9 is the TGA curve of the crystal form C of the compound of formula (I).

FIG. 10 is the DSC curve of the crystal form C of the compound of formula (I).

FIG. 11 is the XRPD pattern of the crystal form D of the compound of formula (I).

FIG. 12 is the TGA curve of the crystal form D of the compound of formula (I).

FIG. 13 is the DSC curve of the crystal form D of the compound of formula (I).

FIG. 14 is the XRPD pattern of the crystal form E of the compound of formula (I).

FIG. 15 is the TGA curve of the crystal form E of the compound of formula (I).

FIG. 16 is the DSC curve of the crystal form E of the compound of formula (I).

FIG. 17 is the XRPD pattern of the crystal form F of the compound of formula (I).

FIG. 18 is the TGA curve of the crystal form F of the compound of formula (I).

FIG. 19 is the DSC curve of the crystal form F of the compound of formula (I).

FIG. 20 is the XRPD pattern of the crystal form G of the compound of formula (I).

FIG. 21 is the XRPD pattern of the crystal form H of the compound of formula (I).

FIG. 22 is the XRPD pattern of the crystal form I of the compound of formula (I).

FIG. 23 is the XRPD pattern of the crystal form J of the compound of formula (I).

FIG. 24 is the XRPD pattern of the crystal form K of the compound of formula (I).

FIG. 25 is the XRPD pattern of the crystal form L of the compound of formula (I).

FIG. 26 is the XRPD pattern of the crystal form M of the compound of formula (I).

FIG. 27 is the XRPD pattern of the crystal form N of the compound of formula (I).

FIG. 28 is the XRPD pattern of the crystal form O of the compound of formula (I).

FIG. 29 is the XRPD pattern of the crystal form P of the compound of formula (I).

FIG. 30 is the XRPD pattern of the crystal form Q of the compound of formula (I).

FIG. 31 is the XRPD pattern of the crystal form R of the compound of formula (I).

FIG. 32 is the XRPD pattern of the crystal form S of the compound of formula (I).

FIG. 33 is the XRPD pattern of the crystal form T of the compound of formula (I).

FIG. 34 is the XRPD pattern of the crystal form U of the compound of formula (I).

FIG. 35 is the XRPD pattern of the crystal form V of the compound of formula (I).

FIG. 36 is the XRPD pattern of the crystal form W of the compound of formula (I).

FIG. 37 is the XRPD pattern of the crystal form X of the compound of formula (I).

FIG. 38 is the XRPD pattern of the crystal form Y of the compound of formula (I).

FIG. 39 is the XRPD pattern of the crystal form Z of the compound of formula (I).

FIG. 40 is the XRPD pattern of the crystal form AA of the compound of formula (I).

FIG. 41 is the XRPD pattern of the crystal form BB of the compound of formula (I).

FIG. 42 is the XRPD pattern of the crystal form CC of the compound of formula (I).

FIG. 43 is the XRPD pattern of the crystal form DD of the compound of formula (I).

FIG. 44 is the XRPD pattern of the crystal form EE of the compound of formula (I).

FIG. 45 is the XRPD pattern of the crystal form FF of the compound of formula (I).

FIG. 46 is the XRPD pattern of the crystal form GG of the compound of formula (I).

FIG. 47 is the XRPD pattern of the crystal form HH of the compound of formula (I).

FIG. 48 is the XRPD pattern of the crystal form II of the compound of formula (I).

FIG. 49 is the XRPD pattern of the crystal form JJ of the compound of formula (I).

FIG. 50 is the XRPD pattern of the crystal form KK of the compound of formula (I).

FIG. 51 is the XRPD pattern of the crystal form LL of the compound of formula (I).

FIG. 52 is the XRPD pattern of the crystal form MM of the compound of formula (I).

FIG. 53 is the XRPD pattern of the crystal form NN of the compound of formula (I).

FIG. 54 is the DVS pattern of the crystal form C of the compound of formula (I).

### DETAILED DESCRIPTION OF THE INVENTION

In order to better understand the content of the present disclosure, the present disclosure is further illustrated below in conjunction with specific examples, but the specific examples are not intended to limit the content of the present disclosure.

### Example 1: Preparation of Compound of Formula (I)

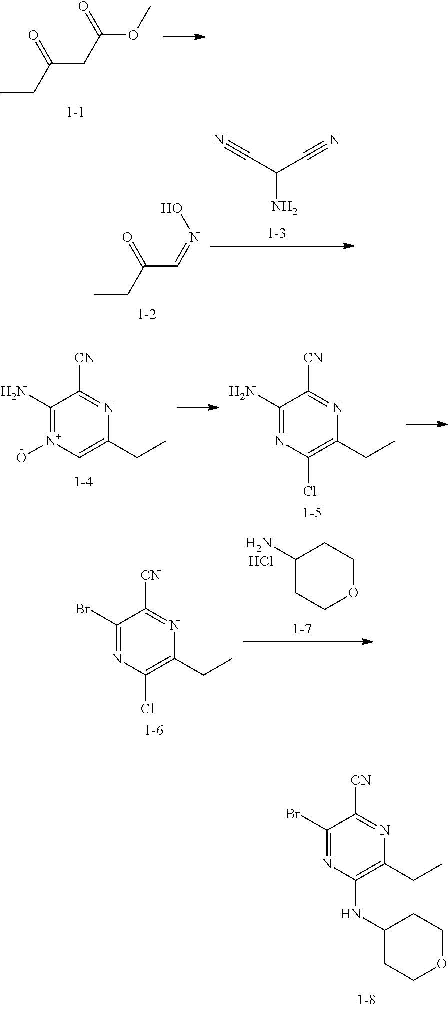

Step A: Compound 1-1 (30 g, 230.52 mmol, 28.57 mL, 1 equiv) was added to water (600 mL), and sodium hydroxide (11.99 g, 299.67 mmol, 1.3 equiv) was then added. The mixture was stirred at 20° C. for 16 hours. The system was cooled to 0° C. to 5° C., and a solution of sodium nitrite (17.50 g, 253.57 mmol, 1.1 equiv) in water (60 mL) was then slowly added. The system was adjusted to a pH of 4 with sulfuric acid, and then further stirred at 20° C. for 12 hours. The aqueous phase was extracted with ethyl acetate (400 mL×2). The organic phases were combined, washed with saturated brine (100 mL×2), dried over sodium sulfate, and concentrated to give compound 1-2. ¹H NMR (400 MHz, CDCl₃) δ=8.83-8.54 (m, 1H), 7.56 (s, 1H), 2.80 (q, J=7.2 Hz, 2H), 1.13 (t, J=7.2 Hz, 3H).

Step B: Compound 1-2 (20 g, 197.82 mmol, 1 equiv) was dissolved in isopropanol (400 mL), and then compound 1-3 (50 g, 197.41 mmol, 0.998 equiv, p-toluenesulfonate) was added. The mixed system was stirred at 20° C. for 16 hours. The reaction solution was poured into water (300 mL), and extracted with ethyl acetate (500 mL×3). The organic phases were combined, washed with saturated brine (800 mL), dried over sodium sulfate, and concentrated to give compound 1-4. MS (ESI) m/z: 165.3 [M+H⁺].

Step C: Compound 1-4 (31 g, 188.84 mmol, 1 equiv) was dissolved in N,N-dimethylformamide (300 mL) and cooled to 0° C. Phosphorus oxychloride (78.52 g, 512.09 mmol, 47.59 mL, 2.71 equiv) was then slowly added dropwise while keeping the temperature below 5° C. After the addition was completed, the system was heated to 80° C. and stirred for 2 hours. The reaction solution was added dropwise to ice (900 g), allowed to warm to 20° C., and then stirred for 16 hours. Solid was precipitated out and filtered. The filter cake was collected and dried under vacuum to give compound 1-5.

Step D: Tert-butyl nitrite (20.61 g, 199.88 mmol, 23.77 mL, 2.5 equiv) and CuBr₂ (21.43 g, 95.94 mmol, 4.49 mL, 1.2 equiv) were dissolved in N,N-dimethyl formamide (200 mL), and the system was heated to 65° C. A solution of compound 1-5 (14.6 g, 79.95 mmol, 1 equiv) in N,N-dimethylformamide (150 mL) was then added dropwise. The reaction solution was reacted at 65° C. for 0.5 hours, and then poured into ice water (1000 g). The precipitated solid was filtered. The filter cake was dissolved in ethyl acetate (300 ml) and filtered again. The filtrate was concentrated to give compound 1-6. ¹H NMR (400 MHz, DMSO-d₆) δ=2.92 (q, J=7.2 Hz, 2H), 1.23 (t, J=7.2 Hz, 3H).

Step E: Compound 1-6 (4 g, 16.23 mmol, 1 equiv) and compound 1-7 (1.97 g, 14.31 mmol, 0.882 equiv) were dissolved in 1,4-dioxane (50 mL), and then N,N-diisopropylethylamine (5.03 g, 38.95 mmol, 6.78 mL, 2.4 equiv) was added. The mixture was heated to 65° C. and stirred for 12 hours. Water (100 ml) was poured into the reaction solution, and the mixture was stirred at 20° C. for 0.5 hour. The mixture was filtered. The filter cake was washed with water, and dried under vacuum to give compound 1-8. MS (ESI) m/z: 310.9, 312.9 [M+H⁺].

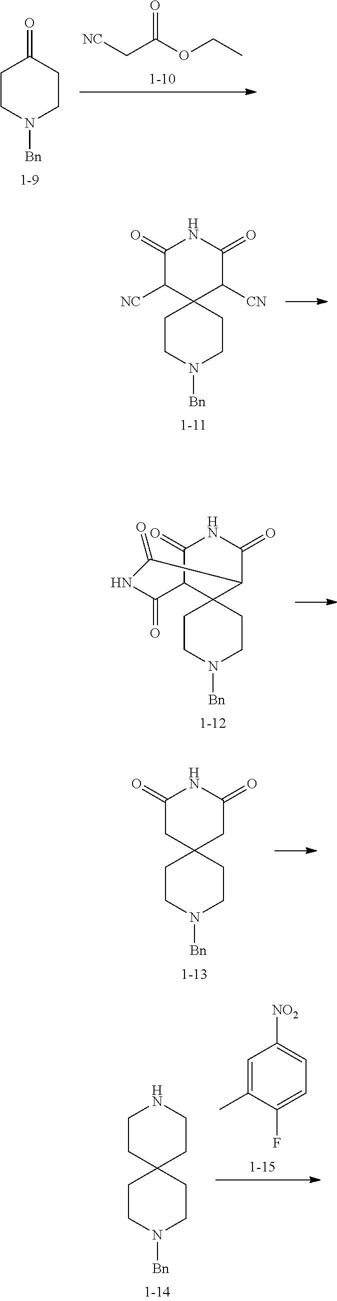

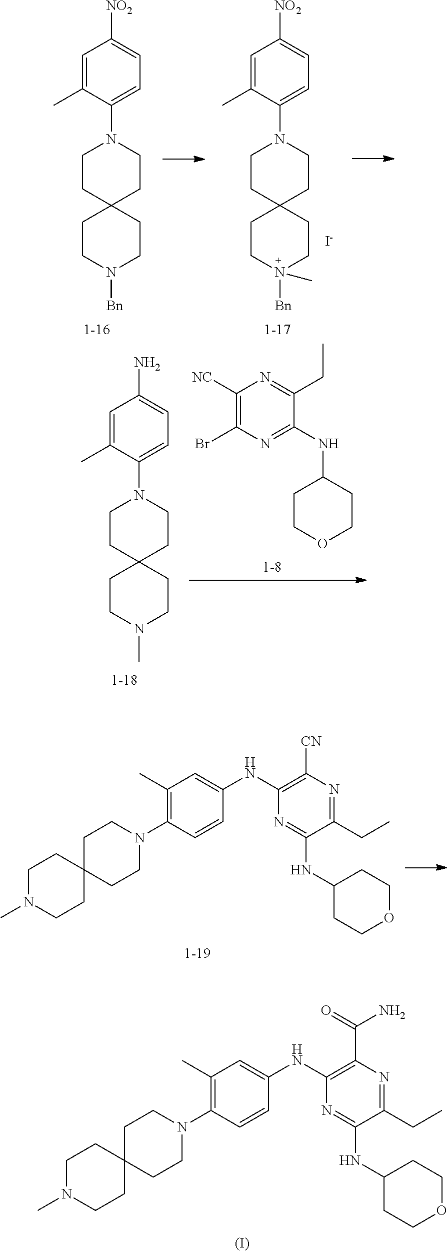

Step F: Ammonium acetate (2.04 g, 26.42 mmol, 0.1 equiv) was added to a solution of compound 1-10 (89.65 g, 792.59 mmol, 84.58 mL, 3 equiv) in methanol (100 mL) at 5° C. to 8° C., and then compound 1-9 (50 g, 264.20 mmol, 49.02 mL, 1 equiv) was added Ammonia water (51.85 g, 369.87 mmol, 56.98 mL, 25%, 1.4 equiv) was then added to the mixed solution below 10° C. The mixed solution was stirred at 0° C. to 5° C. for 1 hour. The reaction mixture was then warmed to 20° C. and stirred for 20 hours. Water (100 ml) was added to the system and the mixture was heated to 55° C. The reaction solution was adjusted to a pH of 4 with hydrochloric acid (12 mol/L) while keeping the temperature not to exceed 70° C. Thereafter, the mixture was cooled to 10° C., stirred for 30 minutes, and then filtered. The filter cake was washed with water and dried under reduced pressure to give compound 1-11. MS (ESI) m/z: 323.1 [M+H⁺].

Step G: To a mixture of sulfuric acid (161.92 g, 1.65 mol, 88 mL, 10.65 equiv) and water (12.00 g, 666.10 mmol, 12 mL, 4.30 equiv) was added compound 1-11 (49.95 g, 154.95 mmol, 1 equiv). At this time, the temperature of the mixture was raised to 40° C. The mixture was then heated to 80° C. and stirred for 2 hours. Water (20.00 g, 1.11 mol, 20 mL, 7.16 equiv) was then added. The mixture was heated to 100° C. and stirred for 1.5 hours. Water (250 ml) was added to the reaction solution and the mixture was stirred at 30° C. for 12 hours. The reaction solution was then filtered. The filter cake was washed with water, and dried under reduced pressure to give compound 1-12. MS (ESI) m/z: 342.0 [M+H⁺].

Step H: To an aqueous solution of sodium hydroxide (5 mol/L, 183.45 mL, 8 equiv) was added compound 1-12 (39.14 g, 114.66 mmol, 1 equiv). The mixture was heated to 80° C. and stirred for 2 hours. The system was cooled to 60° C., and hydrochloric acid (12 mol/L, 75 ml, 7.85 equiv) was slowly added. The system was heated to 75° C. and hydrochloric acid (12 mol/L, 15 ml, 1.57 equiv) was added dropwise. The mixture was heated to 85° C. and stirred for 1 hour. The mixture was then cooled to 25° C. and stirred for 16 hours. Water (200 ml) was added to the reaction solution. The mixture was cooled to 10° C., and filtered. The filter cake was washed with water (300 ml) and dried under reduced pressure to give compound 1-13. MS (ESI) m/z: 273.1 [M+H⁺].

Step I: Compound 1-13 (28 g, 102.81 mmol, 1 equiv) was dissolved in tetrahydrofuran (300 mL). The mixture was heated to 70° C., and lithium tetrahydroaluminum (15.61 g, 411.25 mmol, 4 equiv) was then added in portions to the solution. The mixture was stirred at 70° C. for 12 hours. After cooled to room temperature, a saturated sodium sulfate solution (30 mL) was slowly added dropwise to the reaction solution, and the mixture was then filtered. The filter cake was washed with ethyl acetate (100 mL). The filtrates were combined and concentrated to give compound 1-14. MS (ESI) m/z: 245.1 [M+H⁺].

Step J: Compound 1-14 (0.5 g, 2.05 mmol, 1 equiv) and compound 1-15 (317.39 mg, 2.05 mmol, 1 equiv) were dissolved in N,N-dimethylformamide (10 mL), and potassium carbonate (565.55 mg, 4.09 mmol, 2 equiv) was added. The mixture was heated to 80° C. and stirred for 12 hours. The reaction solution was poured into water (60 mL), and extracted with ethyl acetate (60 mL×2). The organic phases were combined, washed with saturated brine (60 mL), dried and concentrated to give compound 1-16. MS (ESI) m/z: 380.0 [M+H⁺].

Step K: To a solution of compound 1-16 (550 mg, 1.45 mmol, 1 equiv) in dichloromethane (15 mL) was added methyl iodide (246.85 mg, 1.74 mmol, 108.27 μL, 1.2 equiv), and the mixture was stirred at 25° C. for 12 hours. The reaction solution was concentrated to give compound 1-17. MS (ESI) m/z: 394.1 [M+H⁺].

Step L: To a solution of compound 1-17 (620 mg, 1.19 mmol, 1 equiv) in ethanol (20 mL) was added wet palladium carbon (100 mg, 10%). The mixture was purged with hydrogen, and then heated to 60° C. The mixture was stirred under hydrogen pressure of 50 psi for 12 hours. The reaction solution was then filtered and the filtrate was concentrated to give compound 1-18. MS (ESI) m/z: 274.1 [M+H⁺].

Step M: To a solution of compound 1-18 (300 mg, 1.10 mmol, 1 equiv) and compound 1-8 (341.43 mg, 1.10 mmol, 1 equiv) in 1,4-dioxane (10 mL) were added palladium acetate (24.63 mg, 109.72 μmol, 0.1 equiv), 4,5-bis(diphenylphosphino)-9,9-dimethylxanthene (63.49 mg, 109.72 μmol, 0.1 equiv) and potassium carbonate (303.29 mg, 2.19 mmol, 2 equiv). The system was purged with nitrogen, then heated to 80° C., and stirred under a nitrogen atmosphere for 12 hours. The reaction solution was filtered, and the filter cake was washed with ethyl acetate (60 ml). The filtrate was concentrated and the resultant crude product was purified to give compound 1-19. MS (ESI) m/z: 504.2 [M+H⁺].

Step N: Compound 1-19 (200 mg, 397.08 μmol, 1 equiv) was dissolved in dimethyl sulfoxide (2 mL) and ethanol (6 mL), and the system was cooled to 0° C. Sodium hydroxide (4 mol/L, 297.81 μL, 3 equiv) and hydrogen peroxide (135.06 mg, 1.19 mmol, 114.46 μL, 30% pure, 3 equiv) was then added. The reaction solution was allowed to warm to 25° C. and stirred for 12 hours.

Method 1 (Preparation of the trifluoroacetate salt of the compound of formula (I)): The reaction solution obtained in Step N was poured into water (30 ml) and extracted with ethyl acetate (40 ml×3). The organic phases were combined, washed with saturated brine (40 ml), dried over sodium sulfate, and concentrated to give the crude product, which was then separated and purified (Preparative high performance liquid chromatography, column: Phenomenex Synergi C18 150*25*10 microns; mobile phase: [water (0.1% trifluoroacetic acid)-acetonitrile]; acetonitrile %: 10%-37%, 10 minutes) to give the trifluoroacetate salt of the compound of formula (I). ¹H NMR (400 MHz, DMSO-d₆) δ=11.13 (s, 1H), 9.23 (br s, 1H), 7.61-7.40 (m, 3H), 7.28-7.11 (m, 2H), 6.85 (d, J=7.2 Hz, 1H), 4.18-4.06 (m, 1H), 3.99-3.91 (m, 2H), 3.41-3.37 (m, 2H), 3.30-3.27 (m, 2H), 3.13-2.91 (m, 6H), 2.79 (d, J=4.4 Hz, 3H), 2.62-2.55 (m, 2H), 2.33-2.28 (m, 3H), 2.03-1.78 (m, 6H), 1.73-1.44 (m, 6H), 1.19 (t, J=7.2 Hz, 3H). MS (ESI) m/z: 522.0 [M+H⁺].

Method 2 (Preparation of the compound of formula (I)): Water (20 ml) was added to the reaction solution obtained in Step N and stirred for 30 minutes. The mixture was filtered, and the filter cake was washed with water (10 ml). The filter cake was slurried with ethanol (5 ml), filtered, and dried under reduced pressure to give the compound of formula (I). ¹H NMR (400 MHz, DMSO-d₆) δ=11.01 (s, 1H), 7.52 (d, J=2.8 Hz, 1H), 7.43 (d, J=2.4 Hz, 1H), 7.36 (dd, J=8.8 Hz, 2.4 Hz, 1H), 7.20 (d, J=2.4 Hz, 1H), 6.99 (d, J=8.8 Hz, 1H), 6.79 (d, J=7.6 Hz, 1H), 4.15-4.06 (m, 1H), 3.95-3.92 (m, 2H), 3.42-3.39 (m, 2H), 2.74-2.71 (m, 4H), 2.56 (q, J=7.6 Hz, 2H), 2.28-2.25 (m, 4H), 2.22 (s, 3H), 2.14 (s, 3H), 1.88-1.84 (m, 2H), 1.69-1.45 (m, 10H), 1.18 (t, J=7.2 Hz, 3H). MS (ESI) m/z: 522.3 [M+H⁺].

### Example 2: Preparation of the Crystal Form a of the Compound of Formula (I)

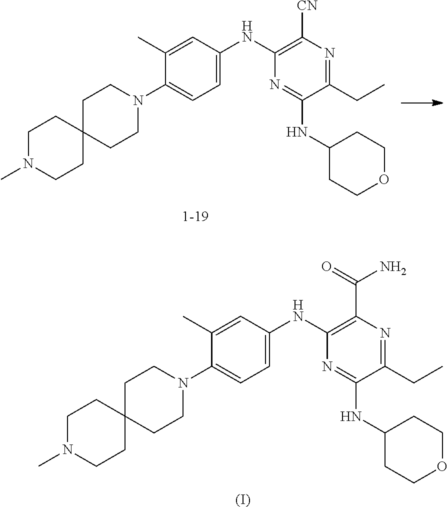 [SMILES: CCc1nc(C#N)c(Nc2ccc(N3CCC4(CCN(C)CC4)CC3)c(C)c2)nc1NC1CCOCC1 ; CCc1nc(C(N)=O)c(Nc2ccc(N3CCC4(CCN(C)CC4)CC3)c(C)c2)nc1NC1CCOCC1 ; "(I)"]

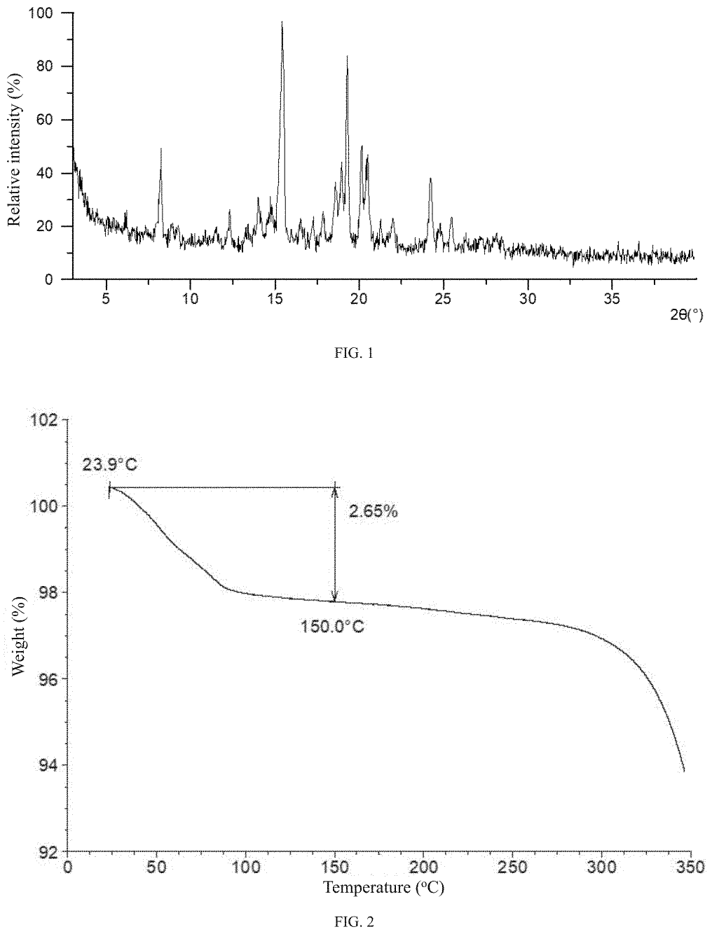

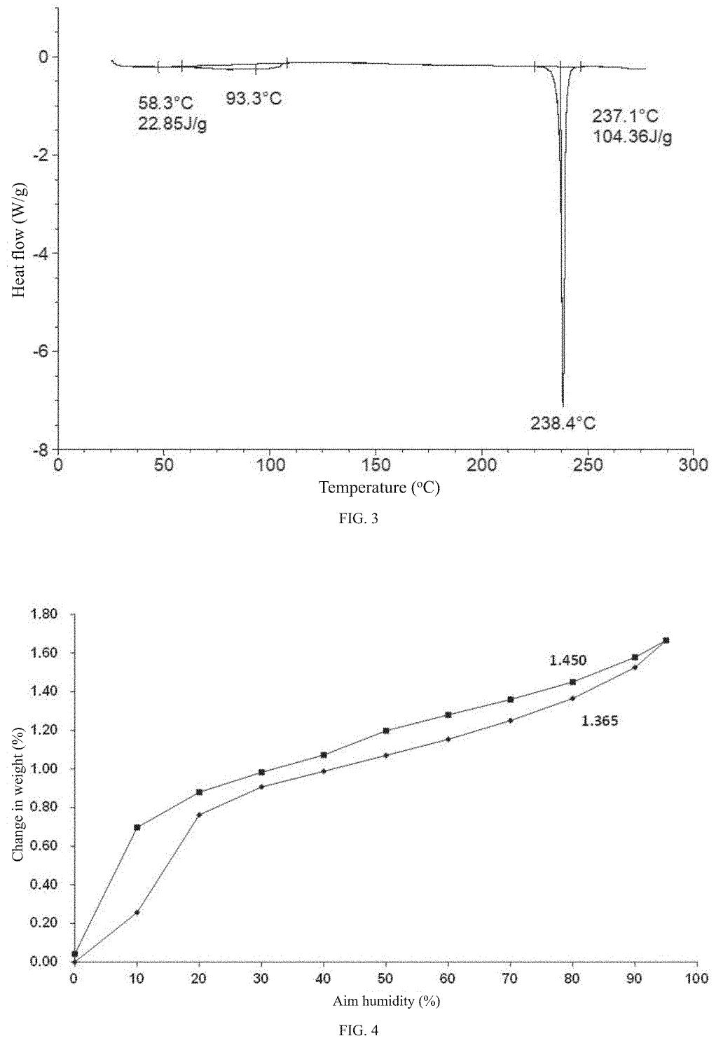

Compound 1-19 (75 g, 145.38 mmol, 1 eq) was added to a solution of DMSO (1500 mL) and EtOH (1500 mL), and the system was cooled down to −10° C. Then NaOH (4 M, 218.06 mL, 6 eq) solution and H₂O₂ (105.95 g, 934.49 mmol, 89.79 mL, 30% purity, 6.43 eq) were added sequentially, while the temperature was kept not higher than 30° C. The reaction solution was then stirred at 25° C. for 12 h. Water (3 L) was added to the reaction solution and the mixture was stirred at 25° C. for 1 h. The reaction solution was filtered and the filter cake was washed with water (1 L) and then dried under reduced pressure. The dried filter cake was slurried sequentially with EtOH/H₂O (1/1, 375 mL), H₂O (375 mL), DCM/heptane (1/3, 375 mL), and acetone (375 mL), and dried under reduced pressure to give the crystal form A of the compound of formula (I). For the crystal form A, the XRPD pattern is shown in FIG. 1, the TGA curve is shown in FIG. 2, the DSC curve is shown in FIG. 3, and the DVS pattern is shown in FIG. 4.

¹H NMR (400 MHz, DMSO-d₆) δ=11.01 (s, 1H), 7.52 (d, J=2.8 Hz, 1H), 7.43 (d, J=2.4 Hz, 1H), 7.36 (dd, J=2.4, 8.8 Hz, 1H), 7.20 (d, J=2.4 Hz, 1H), 6.99 (d, J=8.4 Hz, 1H), 6.79 (d, J=7.6 Hz, 1H), 4.17-4.04 (m, 1H), 3.94 (dd, J=2.8, 11.2 Hz, 2H), 3.45-3.37 (m, 2H), 2.78-2.70 (m, 4H), 2.57 (q, J=7.6 Hz, 2H), 2.31-2.25 (m, 4H), 2.23 (s, 3H), 2.14 (s, 3H), 1.86 (dd, J=2.4, 12.4 Hz, 2H), 1.71-1.43 (m, 10H), 1.18 (t, J=7.6 Hz, 3H)

### Example 3: Preparation of the Crystal Form B of the Compound of Formula (I)

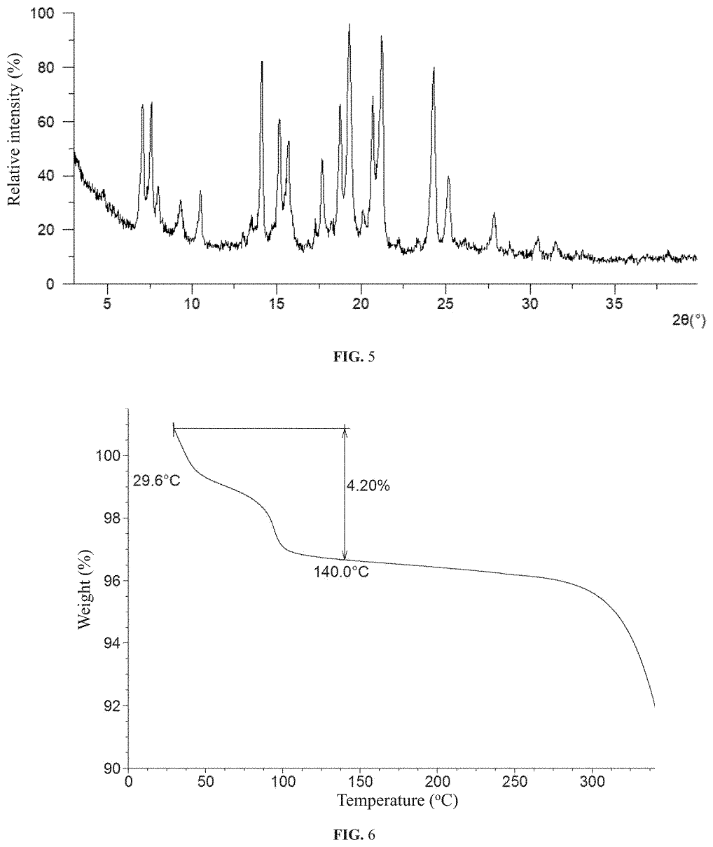

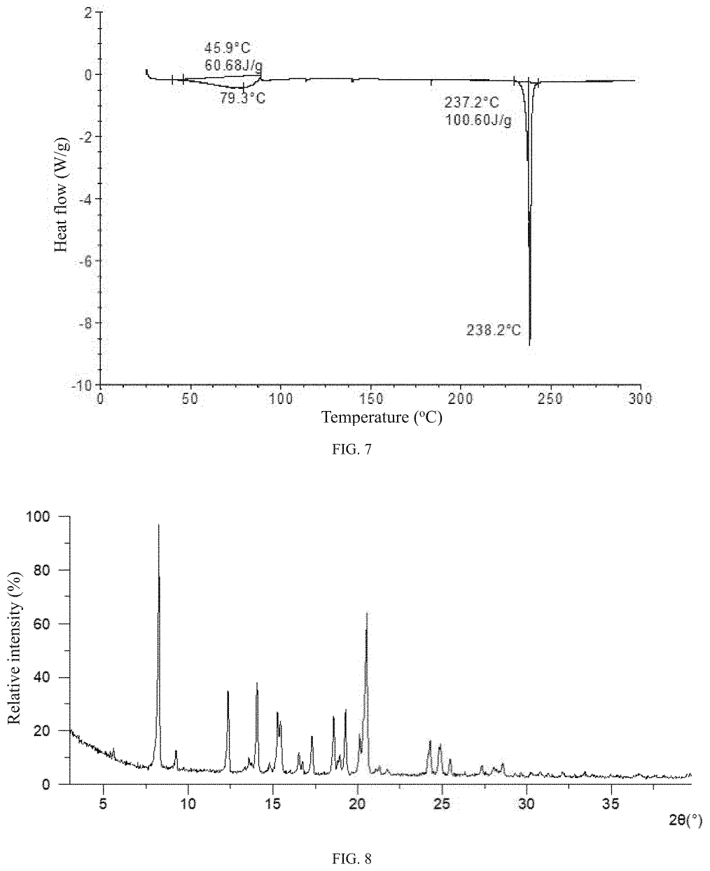

The crystal form A of the compound of formula (I) (100 mg) was added to THF (1 mL). The system was heated to 100° C. and stirred for 1 hour. The heating was stopped, and the system was allowed to naturally cool down to 25° C. The mixture was then stirred at 25° C. for 12 hours. The reaction solution was filtered and the filter cake was dried under reduced pressure to give the crystal form B of the compound of formula (I). For the crystal form B, the XRPD pattern is shown in FIG. 5, the TGA curve is shown in FIG. 6, and the DSC curve is shown in FIG. 7.

¹H NMR (400 MHz, DMSO-d₆) δ=11.02 (s, 1H), 7.52 (d, J=2.4 Hz, 1H), 7.43 (d, J=2.4 Hz, 1H), 7.36 (dd, J=2.4, 8.4 Hz, 1H), 7.22 (d, J=2.4 Hz, 1H), 6.98 (d, J=8.8 Hz, 1H), 6.79 (d, J=7.6 Hz, 1H), 4.17-4.04 (m, 1H), 3.94 (dd, J=2.4, 11.2 Hz, 2H), 3.43-3.37 (m, 2H), 2.77-2.68 (m, 4H), 2.57 (q, J=7.2 Hz, 2H), 2.30-2.25 (m, 4H), 2.23 (s, 3H), 2.14 (s, 3H), 1.86 (dd, J=2.0, 12.4 Hz, 2H), 1.70-1.43 (m, 10H), 1.18 (t, J=7.3 Hz, 3H)

### Example 4: Preparation of the Crystal Form C of the Compound of Formula (I)

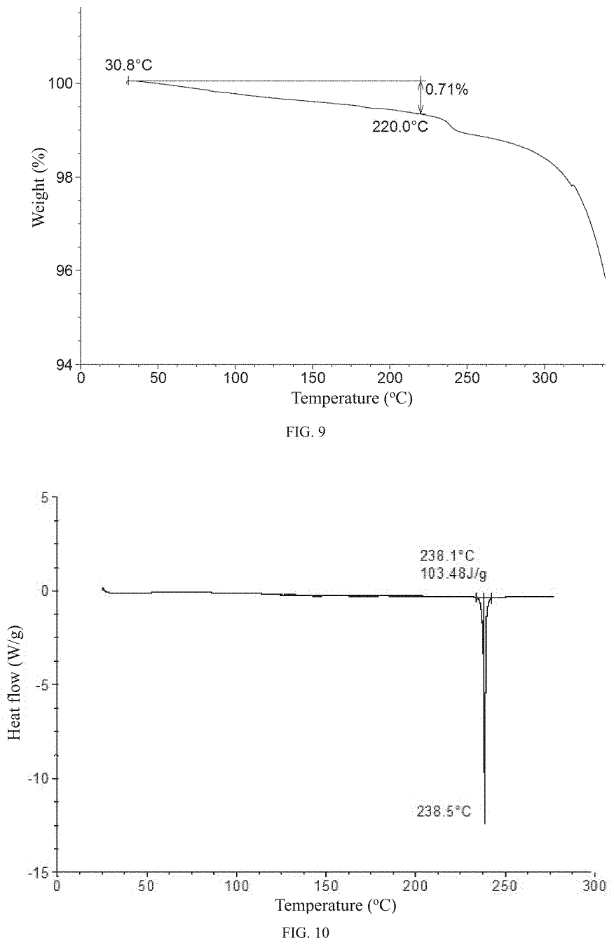

The crystal form A of the compound of formula (I) (1 g) was added to DMSO (5 mL) and acetone (5 mL). The mixture was heated to 100° C. and stirred for 1 hour, and then allowed to naturally cool down to 25° C. and stirred for 12 hours. The above mixture was filtered and the filter cake was dried under vacuum to give the crystal form C of the compound of formula (I). For the crystal form C, the XRPD pattern is shown in FIG. 8, the TGA curve is shown in FIG. 9, the DSC curve is shown in FIG. 10, and the DVS pattern is shown in FIG. 54.

¹H NMR (400 MHz, CHLOROFORM-d) δ=10.69 (s, 1H), 7.53-7.42 (m, 3H), 6.97 (d, J=8.4 Hz, 1H), 5.18 (s, 1H), 4.60 (d, J=7.2 Hz, 1H), 4.28-4.14 (m, 1H), 4.08-4.00 (m, 2H), 3.56 (dt, J=2.0, 11.6 Hz, 2H), 2.86-2.77 (m, 4H), 2.51 (q, J=7.2 Hz, 2H), 2.43-2.38 (m, 4H), 2.30 (s, 6H), 2.11 (br dd, J=2.2, 12.4 Hz, 2H), 1.69-1.51 (m, 10H), 1.30 (t, J=7.2 Hz, 3H)

### Example 5: Preparation of the Crystal Form D of the Compound of Formula (I)

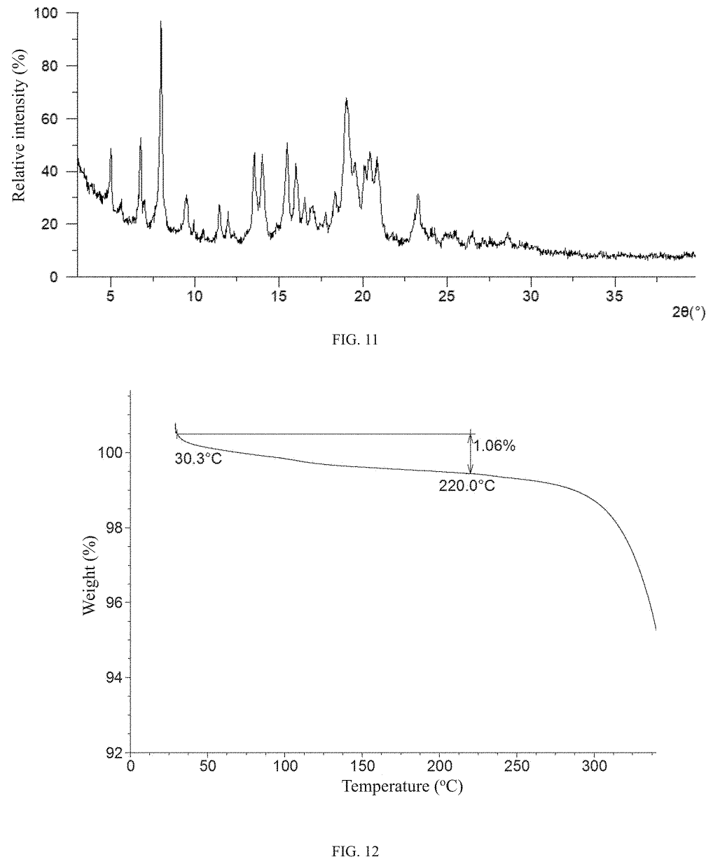

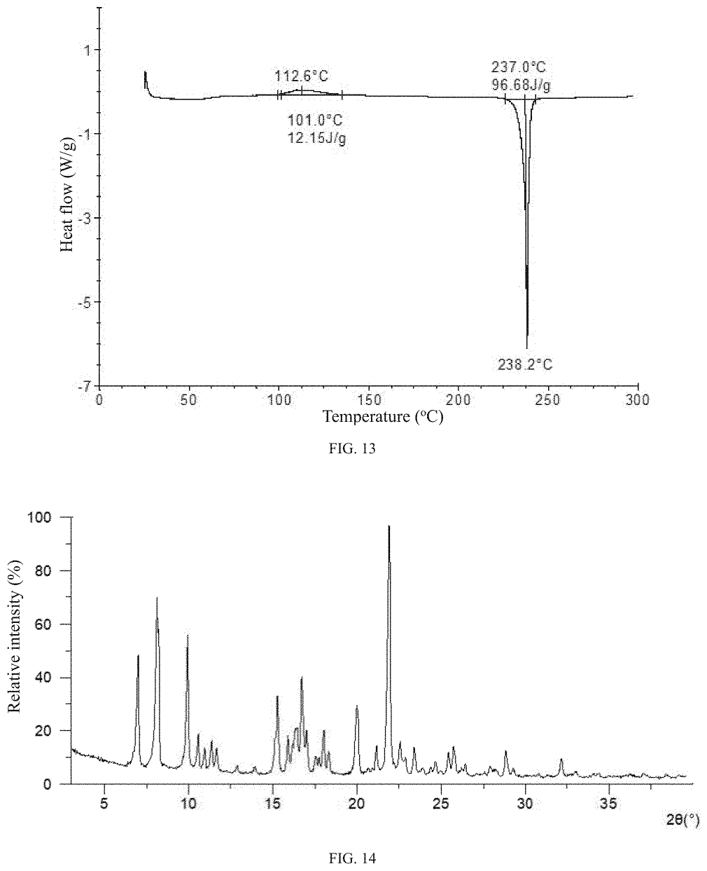

The crystal form C of the compound of formula (I) (200 mg) and methanol (4 mL) were added to a reaction flask. The mixture was heated to 50° C. and stirred for 48 hours. Then the above mixture was filtered and the filter cake was dried under vacuum at 60° C. to give the crystal form D of the compound of formula (I). For the crystal form D, the XRPD pattern is shown in FIG. 11, the TGA curve is shown in FIG. 12, and the DSC curve is shown in FIG. 13.

¹H NMR (400 MHz, DMSO-d₆) δ=11.02 (s, 1H), 7.52 (d, J=2.8 Hz, 1H), 7.43 (d, J=2.4 Hz, 1H), 7.37 (dd, J=2.4, 8.8 Hz, 1H), 7.21 (d, J=2.8 Hz, 1H), 6.99 (d, J=8.8 Hz, 1H), 6.79 (d, J=7.6 Hz, 1H), 4.17-4.04 (m, 1H), 3.94 (dd, J=2.8, 11.4 Hz, 2H), 3.45-3.36 (m, 2H), 2.76-2.69 (m, 4H), 2.58 (q, J=7.6 Hz, 2H), 2.28-2.26 (m, 4H), 2.23 (s, 3H), 2.15 (s, 3H), 1.87 (dd, J=2.0, 12.4 Hz, 2H), 1.71-1.44 (m, 10H), 1.19 (t, J=7.6 Hz, 3H)

### Example 6: Preparation of the Crystal Form E of the Compound of Formula (I)

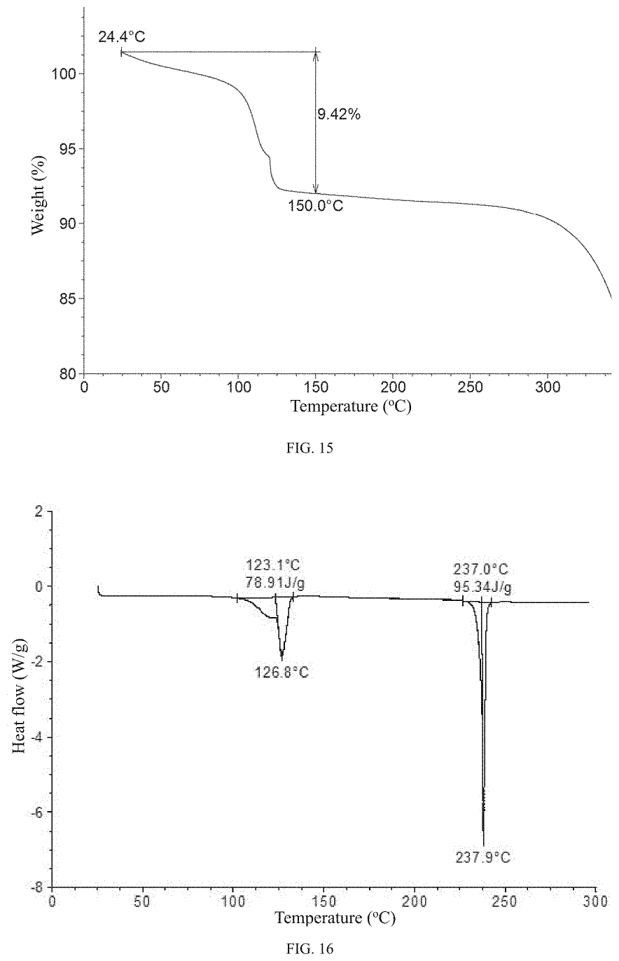

The crystal form C of the compound of formula (I) (200 mg) and ethanol (4 mL) were added to a reaction flask. The mixture was heated to 50° C. and stirred for 48 hours. Then the above mixture was filtered and the filter cake was dried under vacuum at 60° C. to give the crystal form E of the compound of formula (I). For the crystal form E, the XRPD pattern is shown in FIG. 14, the TGA curve is shown in FIG. 15, and the DSC curve is shown in FIG. 16.

¹H NMR (400 MHz, DMSO-d₆) δ=11.03 (s, 1H), 7.52 (d, J=2.4 Hz, 1H), 7.44 (d, J=2.4 Hz, 1H), 7.37 (dd, J=2.4, 8.8 Hz, 1H), 7.22 (d, J=2.4 Hz, 1H), 6.99 (d, J=8.8 Hz, 1H), 6.79 (d, J=7.6 Hz, 1H), 4.36 (t, J=5.2 Hz, 1H), 4.17-4.06 (m, 1H), 3.95 (dd, J=2.8, 11.2 Hz, 2H), 3.48-3.37 (m, 4H), 2.78-2.70 (m, 4H), 2.58 (q, J=7.2 Hz, 2H), 2.27-2.25 (m, 4H), 2.24 (s, 3H), 2.15 (s, 3H), 1.87 (dd, J=2.0, 12.4 Hz, 2H), 1.72-1.45 (m, 10H), 1.19 (t, J=7.2 Hz, 3H), 1.06 (t, J=7.2 Hz, 3H)

### Example 7: Preparation of the Crystal Form F of the Compound of Formula (I)

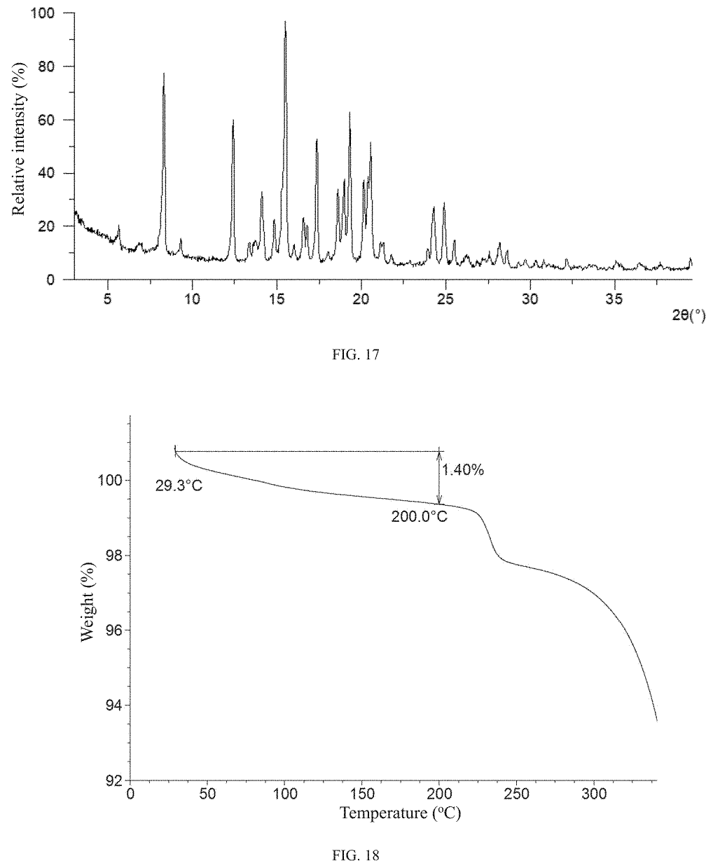

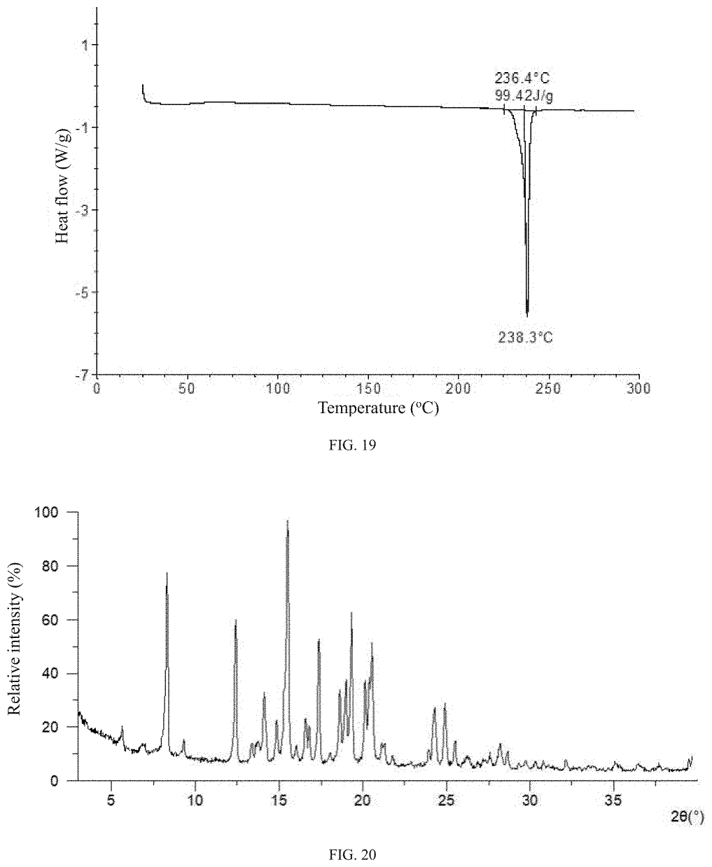

The crystal form C of the compound of formula (I) (200 mg) and 2-MeTHF (4 mL) were added to a reaction flask. The mixture was heated to 50° C. and stirred for 48 hours. Then the above mixture was filtered and the filter cake was dried under vacuum at 60° C. to give the crystal form F of the compound of formula (I). For the crystal form F, the XRPD pattern is shown in FIG. 17, the TGA curve is shown in FIG. 18, and the DSC curve is shown in FIG. 19.

¹H NMR (400 MHz, DMSO-d₆) δ=11.03 (s, 1H), 7.52 (s, 1H), 7.44 (d, J=2.0 Hz, 1H), 7.37 (dd, J=2.4, 8.5 Hz, 1H), 7.22 (s, 1H), 6.99 (d, J=8.8 Hz, 1H), 6.79 (d, J=7.6 Hz, 1H), 4.19-4.06 (m, 1H), 3.95 (dd, J=2.4, 10.8 Hz, 2H), 3.41 (t, J=11.2 Hz, 2H), 2.77-2.70 (m, 4H), 2.58 (q, J=7.2 Hz, 2H), 2.28-2.25 (m, 4H), 2.24 (s, 3H), 2.15 (s, 3H), 1.91-1.83 (m, 2H), 1.71-1.44 (m, 10H), 1.19 (t, J=7.3 Hz, 3H).

### Example 8: Preparation of the Crystal Form G of the Phosphate Salt of the Compound of Formula (I)

The crystal form A of the compound of formula (I) (100 mg) and acetonitrile (3 mL) were added to a reaction flask and the mixture was heated to 80° C. with stirring. Then a mixture of phosphoric acid (19.65 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form G of the phosphate salt of the compound of formula (I). For the crystal form G, the XRPD pattern is shown in FIG. 20.

¹H NMR (400 MHz, DMSO-d₆) δ=11.03 (s, 1H), 7.52 (d, J=2.8 Hz, 1H), 7.44 (d, J=2.4 Hz, 1H), 7.37 (dd, J=2.4, 8.8 Hz, 1H), 7.22 (d, J=2.8 Hz, 1H), 7.00 (d, J=8.8 Hz, 1H), 6.80 (d, J=7.6 Hz, 1H), 4.16-4.05 (m, 1H), 3.94 (dd, J=2.8, 11.6 Hz, 2H), 3.44-3.37 (m, 2H), 2.79-2.70 (m, 4H), 2.65-2.54 (m, 6H), 2.37 (s, 3H), 2.24 (s, 3H), 1.87 (dd, J=2.0, 12.8 Hz, 2H), 1.71-1.47 (m, 10H), 1.18 (t, J=7.2 Hz, 3H).

### Example 9: Preparation of the Crystal Form H of the Hydrochloride Salt of the Compound of Formula (I)

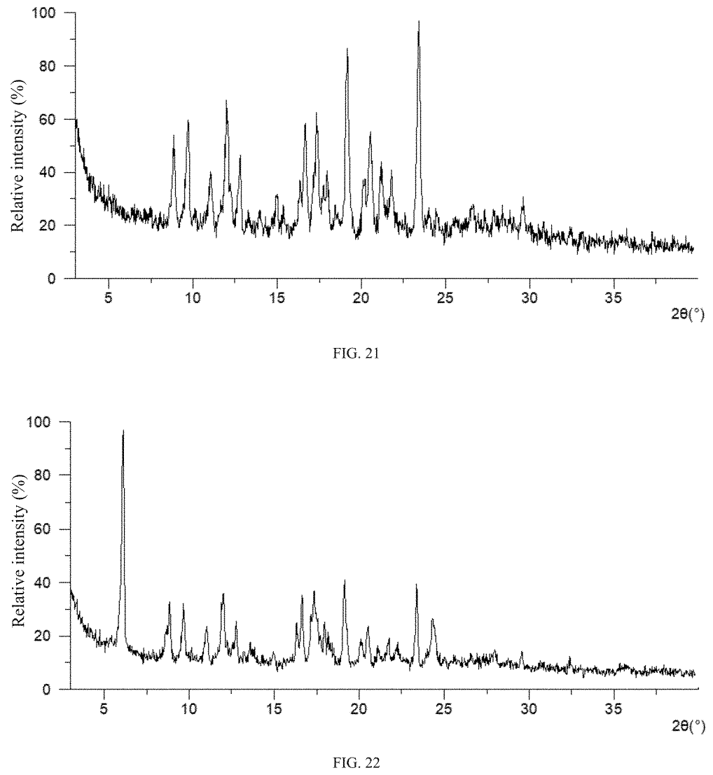

The crystal form A of the compound of formula (I) (100 mg) and THF (2 mL) were added to a reaction flask and the mixture was heated to 80° C. with stirring. Then a mixture of hydrochloric acid (18 μL) and THF (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form H of the hydrochloride salt of the compound of formula (I). For the crystal form H, the XRPD pattern is shown in FIG. 21.

¹H NMR (400 MHz, DMSO-d₆) δ=11.03 (s, 1H), 9.68 (brs, 1H), 7.53 (d, J=2.8 Hz, 1H), 7.45 (d, J=2.4 Hz, 1H), 7.38 (dd, J=2.4, 8.8 Hz, 1H), 7.22 (d, J=2.8 Hz, 1H), 7.01 (d, J=8.8 Hz, 1H), 6.81 (d, J=8.8 Hz, 1H), 4.17-4.06 (m, 1H), 3.99-3.91 (m, 2H), 3.43-3.36 (m, 2H), 3.23-2.98 (m, 4H), 2.78-2.71 (m, 7H), 2.58 (q, J=7.6 Hz, 2H), 2.24 (s, 3H), 1.90-1.83 (m, 2H), 1.82-1.44 (m, 10H), 1.19 (t, J=7.6 Hz, 3H)

### Example 10: Preparation of the Crystal Form I of the Hydrochloride Salt of the Compound of Formula (I)

The crystal form A of the compound of formula (I) (100 mg) and isopropyl alcohol (3 mL) were added to a reaction flask and the mixture was heated to 80° C. with stirring. Then a mixture of hydrochloric acid (18 μL) and isopropyl alcohol (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form I of the hydrochloride salt of the compound of formula (I). For the crystal form I, the XRPD pattern is shown in FIG. 22.

¹H NMR (400 MHz, DMSO-d₆) δ=11.04 (s, 1H), 9.75 (brs, 1H), 7.53 (d, J=2.4 Hz, 1H), 7.45 (d, J=2.4 Hz, 1H), 7.38 (dd, J=2.4, 8.8 Hz, 1H), 7.22 (d, J=2.8 Hz, 1H), 7.01 (d, J=8.8 Hz, 1H), 6.81 (d, J=7.6 Hz, 1H), 4.17-4.05 (m, 1H), 3.96-3.93 (m, 2H), 3.44-3.36 (m, 2H), 3.25-2.99 (m, 4H), 2.79-2.71 (m, 7H), 2.58 (q, J=7.6 Hz, 2H), 2.24 (s, 3H), 1.89-1.46 (m, 12H), 1.18 (t, J=7.2 Hz, 3H).

### Example 11: Preparation of the Crystal Form J of the Hydrochloride Salt of the Compound of Formula (I)

The crystal form A of the compound of formula (I) (100 mg) and acetonitrile (3 mL) were added to a reaction flask and the mixture was heated to 80° C. with stirring. Then a mixture of hydrochloric acid (18 μL) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form J of the hydrochloride salt of the compound of formula (I). For the crystal form J, the XRPD pattern is shown in FIG. 23.

¹H NMR (400 MHz, DMSO-d₆) δ=11.04 (s, 1H), 9.90-9.77 (br s, 1H), 7.57-7.43 (m, 2H), 7.39 (d, J=8.0 Hz, 1H), 7.23 (s, 1H), 7.01 (d, J=8.0 Hz, 1H), 6.82 (d, J=6.8 Hz, 1H), 4.21-4.05 (m, 1H), 3.95 (dd, J=2.8, 10.8 Hz, 2H), 3.45-3.37 (m, 2H), 3.26-3.23 (m, 2H), 3.12-2.99 (m, 2H), 2.79-2.71 (m, 7H), 2.58 (q, J=7.6 Hz, 2H), 2.25 (s, 3H), 1.96-1.46 (m, 12H), 1.19 (t, J=7.2 Hz, 3H).

### Example 12: Preparation of the Crystal Form K of the Sulfate Salt of the Compound of Formula (I)

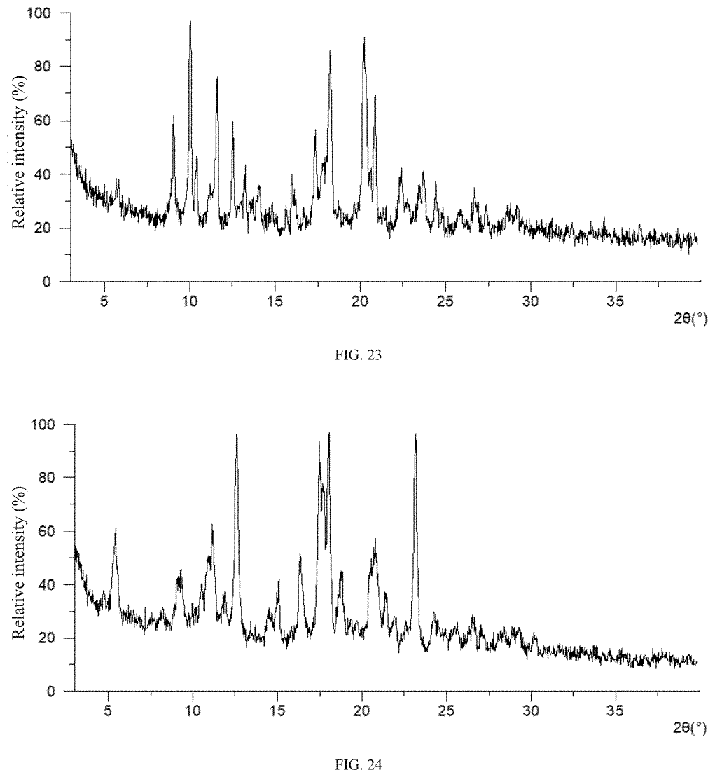

The crystal form A of the compound of formula (I) (100 mg) and acetonitrile (3 mL) were added to a reaction flask and the mixture was heated to 80° C. with stirring. Then a mixture of sulfuric acid (12.19 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form K of the sulfate salt of the compound of formula (I). For the crystal form K, the XRPD pattern is shown in FIG. 24.

¹H NMR (400 MHz, DMSO-d₆) δ=11.03 (s, 1H), 7.53 (d, J=2.8 Hz, 1H), 7.44 (d, J=2.4 Hz, 1H), 7.38 (dd, J=2.4, 8.4 Hz, 1H), 7.22 (d, J=2.8 Hz, 1H), 7.00 (d, J=8.8 Hz, 1H), 6.80 (d, J=7.6 Hz, 1H), 4.17-4.04 (m, 1H), 3.94 (dd, J=3.2, 11.2 Hz, 2H), 3.44-3.37 (m, 2H), 2.82-2.71 (m, 7H), 2.58 (q, J=7.6 Hz, 2H), 2.53-2.51 (m, 4H), 2.24 (s, 3H), 1.87 (dd, J=2.4, 12.4 Hz, 2H), 1.71-1.52 (m, 10H), 1.19 (t, J=7.6 Hz, 3H)

### Example 13: Preparation of the Crystal Form L of the p-Toluenesulfonate Salt of the Compound of Formula (I)

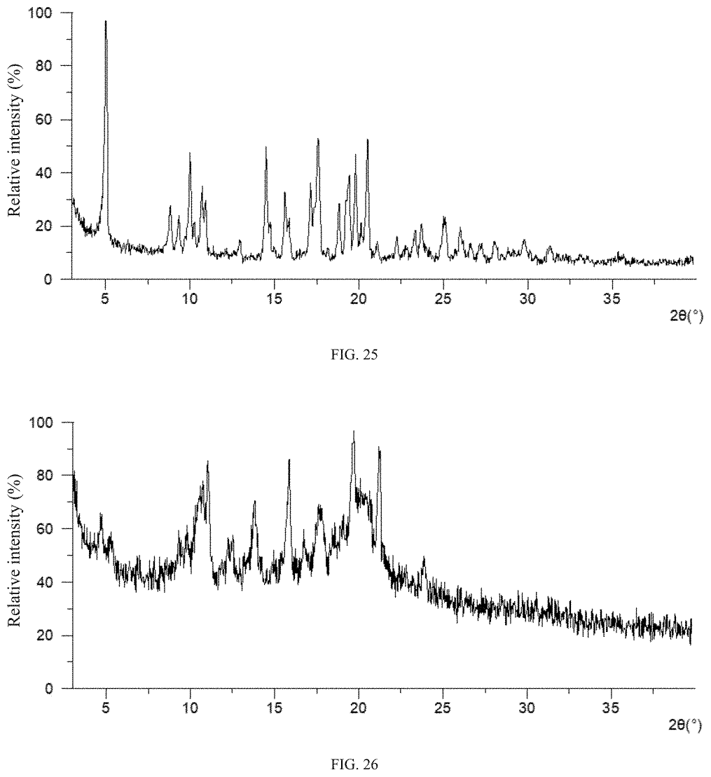

The crystal form A of the compound of formula (I) (100 mg) and acetonitrile (3 mL) were added to a reaction flask and the mixture was heated to 80° C. with stirring. Then a mixture of p-toluenesulfonic acid (35.41 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form L of the p-toluenesulfonate salt of the compound of formula (I). For the crystal form L, the XRPD pattern is shown in FIG. 25.

¹H NMR (400 MHz, DMSO-d₆) δ=11.04 (s, 1H), 7.53 (d, J=2.8 Hz, 1H), 7.50-7.44 (m, 3H), 7.39 (dd, J=2.4, 8.8 Hz, 1H), 7.22 (d, J=2.8 Hz, 1H), 7.11 (d, J=8.0 Hz, 2H), 7.00 (d, J=8.8 Hz, 1H), 6.81 (d, J=7.6 Hz, 1H), 4.16-4.05 (m, 1H), 3.95 (dd, J=2.8, 11.2 Hz, 2H), 3.44-3.36 (m, 2H), 3.19-3.08 (m, 4H), 2.80-2.71 (m, 7H), 2.58 (q, J=7.2 Hz, 2H), 2.28 (s, 3H), 2.24 (s, 3H), 1.95-1.38 (m, 12H), 1.19 (t, J=7.2 Hz, 3H)

### Example 14: Preparation of the Crystal Form M of the Citrate Salt of the Compound of Formula (I)

The crystal form A of the compound of formula (I) (100 mg) and THF (2 mL) were added to a reaction flask and the mixture was heated to 80° C. with stirring. Then a mixture of citric acid (20.40 mg) and THF (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form M of the citrate salt of the compound of formula (I). For the crystal form M, the XRPD pattern is shown in FIG. 26.

¹H NMR (400 MHz, DMSO-d₆) δ=11.02 (s, 1H), 7.53 (d, J=2.8 Hz, 1H), 7.44 (d, J=2.4 Hz, 1H), 7.40-7.35 (m, 1H), 7.21 (d, J=2.4 Hz, 1H), 7.00 (d, J=8.8 Hz, 1H), 6.80 (d, J=7.6 Hz, 1H), 4.17-4.06 (m, 1H), 3.96-3.92 (m, 2H), 3.43-3.37 (m, 2H), 2.78-2.69 (m, 7H), 2.61-2.55 (m, 4H), 2.48-2.44 (m, 3H), 2.24 (s, 3H), 1.88-1.85 (m, 2H), 1.70-1.55 (m, 10H), 1.19 (t, J=7.4 Hz, 3H)

### Example 15: Preparation of the Crystal Form N of the Citrate Salt of the Compound of Formula (I)

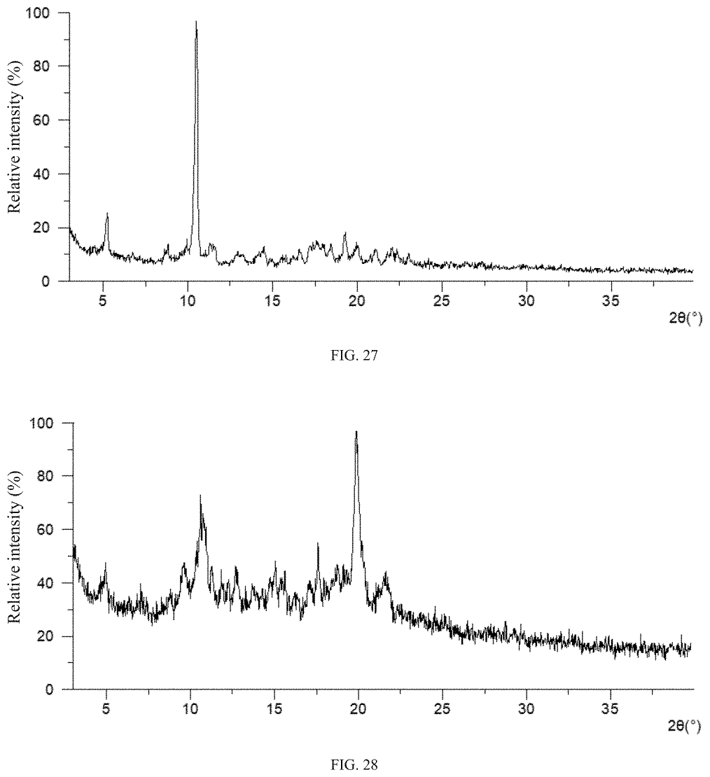

The crystal form A of the compound of formula (I) (100 mg) and isopropyl alcohol (3 mL) were added to a reaction flask and the mixture was heated to 80° C. with stirring. Then a mixture of citric acid (21.05 mg) and isopropyl alcohol (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form N of the citrate salt of the compound of formula (I). For the crystal form N, the XRPD pattern is shown in FIG. 27.

¹H NMR (400 MHz, DMSO-d₆) δ=11.03 (s, 1H), 7.52 (d, J=2.8 Hz, 1H), 7.44 (d, J=2.4 Hz, 1H), 7.38 (dd, J=2.4, 8.8 Hz, 1H), 7.25-7.19 (m, 1H), 7.00 (d, J=8.8 Hz, 1H), 6.80 (d, J=7.6 Hz, 1H), 4.18-4.04 (m, 1H), 3.94 (br dd, J=2.8, 11.2 Hz, 2H), 3.47-3.35 (m, 2H), 2.81-2.66 (m, 8H), 2.62-2.55 (m, 2H), 2.49-2.43 (m, 4H), 2.24 (s, 3H), 1.92-1.82 (m, 2H), 1.69-1.58 (s, 10H), 1.18 (t, J=7.2 Hz, 3H).

### Example 16: Preparation of the Crystal Form O of the Citrate Salt of the Compound of Formula (I)

The crystal form A of the compound of formula (I) (100 mg) and acetonitrile (3 mL) were added to a reaction flask and the mixture was heated to 80° C. with stirring. Then a mixture of citric acid (21.49 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form O of the citrate salt of the compound of formula (I). For the crystal form 0, the XRPD pattern is shown in FIG. 28.

¹H NMR (400 MHz, DMSO-d₆) δ=11.03 (s, 1H), 7.53 (d, J=2.8 Hz, 1H), 7.44 (d, J=2.4 Hz, 1H), 7.38 (dd, J=2.4, 8.8 Hz, 1H), 7.22 (d, J=2.8 Hz, 1H), 7.00 (d, J=8.8 Hz, 1H), 6.80 (d, J=7.6 Hz, 1H), 4.17-4.04 (m, 1H), 3.94 (dd, J=3.2, 11.2 Hz, 2H), 3.43-3.37 (m, 2H), 2.79-2.68 (m, 8H), 2.62-2.55 (m, 2H), 2.49-2.43 (m, 4H), 2.24 (s, 3H), 1.87 (dd, J=2.4, 12.4 Hz, 2H), 1.72-1.55 (m, 10H), 1.19 (t, J=7.2 Hz, 3H)

### Example 17: Preparation of the Crystal Form P of the Citrate Salt of the Compound of Formula (I)

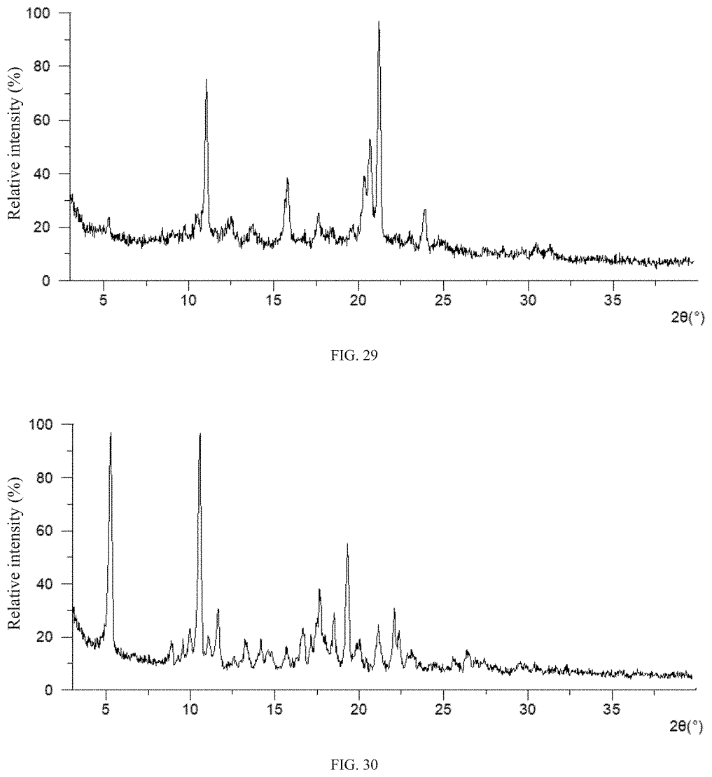

The crystal form A of the compound of formula (I) (100 mg) and THF (2 mL) were added to a reaction flask and the mixture was heated to 80° C. with stirring. Then a mixture of citric acid (38.70 mg) and THF (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form P of the citrate salt of the compound of formula (I). For the crystal form P, the XRPD pattern is shown in FIG. 29.

¹H NMR (400 MHz, DMSO-d₆) δ=11.03 (s, 1H), 7.52 (d, J=2.8 Hz, 1H), 7.44 (d, J=2.4 Hz, 1H), 7.40-7.35 (m, 1H), 7.21 (d, J=2.8 Hz, 1H), 7.02-6.97 (m, 1H), 6.80 (d, J=7.6 Hz, 1H), 4.16-4.05 (m, 1H), 3.96-3.92 (m, 2H), 3.43-3.39 (m, 2H), 2.80-2.69 (m, 8H), 2.61-2.55 (m, 6H), 2.48-2.44 (m, 2H) 2.23 (s, 3H), 1.92-1.82 (m, 2H), 1.70-1.55 (m, 10H), 1.19 (t, J=7.6 Hz, 3H)

### Example 18: Preparation of the Crystal Form Q of the Citrate Salt of the Compound of Formula (I)

The crystal form A of the compound of formula (I) (100 mg) and isopropyl alcohol (3 mL) were added to a reaction flask and the mixture was heated to 80° C. with stirring. Then a mixture of citric acid (39.07 mg) and isopropyl alcohol (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form Q of the citrate salt of the compound of formula (I). For the crystal form Q, the XRPD pattern is shown in FIG. 30.

¹H NMR (400 MHz, DMSO-d₆) δ=11.03 (s, 1H), 7.52 (d, J=2.8 Hz, 1H), 7.44 (d, J=2.4 Hz, 1H), 7.38 (dd, J=2.4, 8.6 Hz, 1H), 7.22 (d, J=2.8 Hz, 1H), 7.00 (d, J=8.8 Hz, 1H), 6.80 (d, J=7.6 Hz, 1H), 4.17-4.05 (m, 1H), 3.94 (br dd, J=2.8, 11.6 Hz, 2H), 3.45-3.37 (m, 2H), 2.83-2.68 (m, 8H), 2.60-2.51 (m, 7H), 2.48-2.43 (m, 2H), 2.24 (s, 3H), 1.87 (br dd, J=2.1, 12.3 Hz, 2H), 1.73-1.50 (m, 10H), 1.18 (t, J=7.2 Hz, 3H).

### Example 19: Preparation of the Crystal Form R of the Citrate Salt of the Compound of Formula (I)

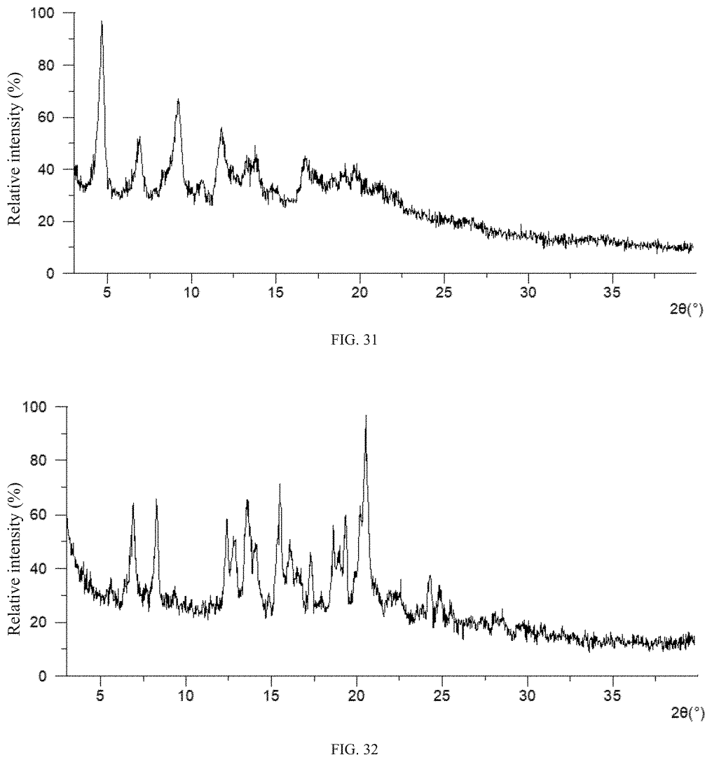

The crystal form A of the compound of formula (I) (100 mg) and acetonitrile (3 mL) were added to a reaction flask and the mixture was heated to 80° C. with stirring. Then a mixture of citric acid (38.42 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form R of the citrate salt of the compound of formula (I). For the crystal form R, the XRPD pattern is shown in FIG. 31.

¹H NMR (400 MHz, DMSO-d₆) δ=11.04 (s, 1H), 7.53 (d, J=2.4 Hz, 1H), 7.45 (d, J=2.4 Hz, 1H), 7.38 (dd, J=2.4, 8.8 Hz, 1H), 7.22 (d, J=2.8 Hz, 1H), 7.00 (d, J=8.8 Hz, 1H), 6.81 (d, J=7.6 Hz, 1H), 4.17-4.05 (m, 1H), 3.95 (dd, J=2.8, 11.2 Hz, 2H), 3.44-3.37 (m, 2H), 3.05-3.01 (m, 4H), 2.80-2.72 (m, 4H), 2.68 (s, 3H), 2.61-2.52 (m, 4H), 2.24 (s, 3H), 1.87 (dd, J=2.0, 12.4 Hz, 2H), 1.76-1.52 (m, 10H), 1.19 (t, J=7.2 Hz, 3H)

### Example 20: Preparation of the Crystal Form S of the Maleate Salt of the Compound of Formula (I)

The crystal form A of the compound of formula (I) (100 mg) and isopropyl alcohol (3 mL) were added to a reaction flask and the mixture was heated to 80° C. with stirring. Then a mixture of maleic acid (12.60 mg) and isopropyl alcohol (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form S of the maleate salt of the compound of formula (I). For the crystal form S, the XRPD pattern is shown in FIG. 32.

¹H NMR (400 MHz, DMSO-d₆) δ=11.03 (s, 1H), 7.52 (d, J=2.8 Hz, 1H), 7.44 (d, J=2.4 Hz, 1H), 7.38 (dd, J=2.4, 8.8 Hz, 1H), 7.22 (d, J=2.4 Hz, 1H), 7.00 (d, J=8.8 Hz, 1H), 6.80 (d, J=7.6 Hz, 1H), 6.01 (s, 1H), 4.16-4.06 (m, 1H), 3.94 (dd, J=2.8, 11.2 Hz, 2H), 3.44-3.37 (m, 2H), 2.79-2.69 (m, 7H), 2.58 (q, J=7.6 Hz, 2H), 2.49-2.44 (m, 4H), 2.24 (s, 3H), 1.87 (dd, J=2.4, 12.6 Hz, 2H), 1.70-1.52 (m, 10H), 1.19 (t, J=7.6 Hz, 3H)

### Example 21: Preparation of the Crystal Form T of the Maleate Salt of the Compound of Formula (I)

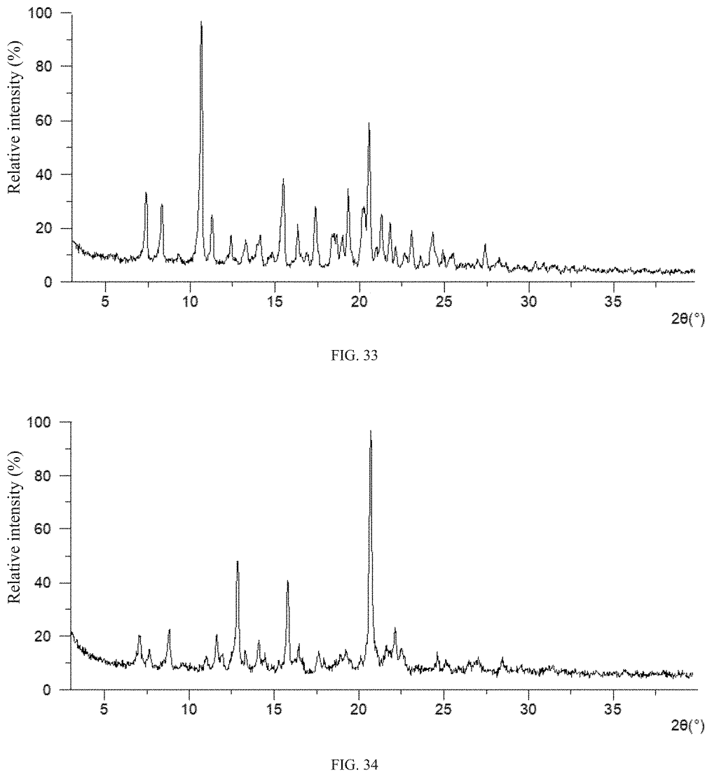

The crystal form A of the compound of formula (I) (100 mg) and acetonitrile (3 mL) were added to a reaction flask and the mixture was heated to 80° C. with stirring. Then a mixture of maleic acid (12.22 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form T of the maleate salt of the compound of formula (I). For the crystal form T, the XRPD pattern is shown in FIG. 33.

¹H NMR (400 MHz, DMSO-d₆) δ=11.03 (s, 1H), 7.53 (d, J=2.8 Hz, 1H), 7.44 (d, J=2.4 Hz, 1H), 7.38 (dd, J=2.4, 8.4 Hz, 1H), 7.22 (br d, J=2.8 Hz, 1H), 7.00 (d, J=8.8 Hz, 1H), 6.80 (d, J=7.6 Hz, 1H), 6.01 (s, 1H), 4.17-4.06 (m, 1H), 3.94 (dd, J=2.8, 11.2 Hz, 2H), 3.43-3.39 (m, 2H), 2.82-2.69 (m, 7H), 2.58 (q, J=7.2 Hz, 2H), 2.53-2.51 (m, 4H), 2.24 (s, 3H), 1.87 (dd, J=2.0, 12.4 Hz, 2H), 1.72-1.53 (m, 10H), 1.19 (t, J=7.2 Hz, 3H)

### Example 22: Preparation of the Crystal Form U of the Maleate Salt of the Compound of Formula (I)

The crystal form A of the compound of formula (I) (100 mg) and THF (2 mL) were added to a reaction flask and the mixture was heated to 80° C. with stirring. Then a mixture of maleic acid (23.60 mg) and THF (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form U of the maleate salt of the compound of formula (I). For the crystal form U, the XRPD pattern is shown in FIG. 34.

¹H NMR (400 MHz, DMSO-d₆) δ=11.04 (s, 1H), 7.53 (d, J=2.4 Hz, 1H), 7.45 (d, J=2.2 Hz, 1H), 7.39 (dd, J=2.4, 8.8 Hz, 1H), 7.22 (d, J=2.4 Hz, 1H), 7.01 (d, J=8.8 Hz, 1H), 6.81 (d, J=7.6 Hz, 1H), 6.03-5.99 (m, 2H), 4.16-4.06 (m, 1H), 3.96-3.92 (m, 2H), 3.44-3.36 (m, 3H), 3.21-3.03 (m, 4H), 2.81-2.71 (m, 7H), 2.62-2.55 (m, 2H), 2.24 (s, 3H), 1.89-1.85 (m, 2H), 1.82-1.34 (m, 10H), 1.19 (t, J=7.6 Hz, 3H)

### Example 23: Preparation of the Crystal Form V of the Maleate Salt of the Compound of Formula (I)

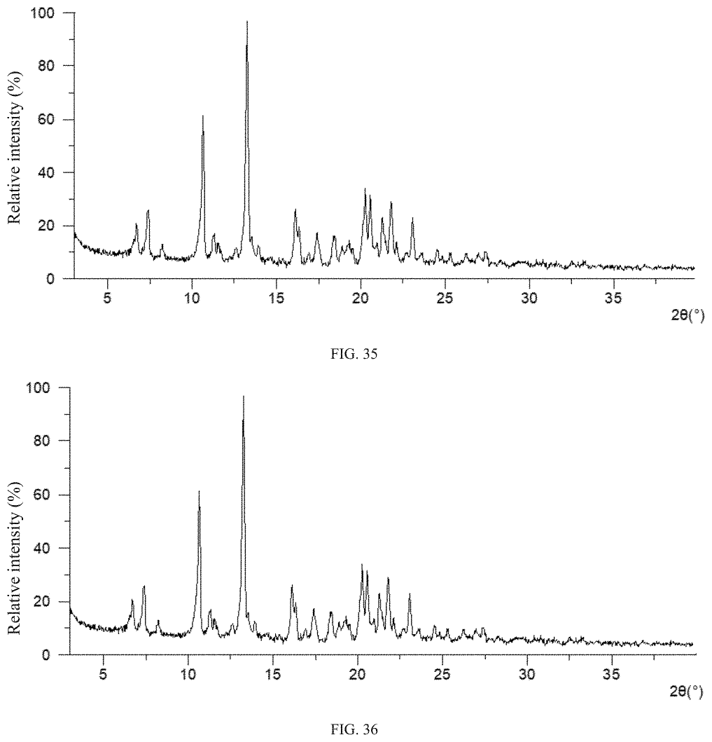

The crystal form A of the compound of formula (I) (100 mg) and acetonitrile (3 mL) were added to a reaction flask and the mixture was heated to 80° C. with stirring. Then a mixture of maleic acid (23.42 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form V of the maleate salt of the compound of formula (I). For the crystal form V, the XRPD pattern is shown in FIG. 35.

¹H NMR (400 MHz, DMSO-d₆) δ=11.04 (s, 1H), 7.53 (d, J=2.4 Hz, 1H), 7.45 (d, J=2.4 Hz, 1H), 7.39 (dd, J=2.4, 8.4 Hz, 1H), 7.22 (d, J=2.4 Hz, 1H), 7.01 (d, J=8.8 Hz, 1H), 6.81 (d, J=7.6 Hz, 1H), 6.01 (s, 2H), 4.17-4.06 (m, 1H), 3.99-3.91 (m, 2H), 3.44-3.36 (m, 2H), 3.25-3.00 (m, 4H), 2.83-2.72 (m, 7H), 2.58 (q, J=7.2 Hz, 2H), 2.24 (s, 3H), 1.90-1.43 (m, 12H), 1.19 (t, J=7.2 Hz, 3H).

### Example 24: Preparation of the Crystal Form W of the Fumarate Salt of the Compound of Formula (I)

The crystal form A of the compound of formula (I) (100 mg) and THF (2 mL) were added to a reaction flask and the mixture was heated to 80° C. with stirring. Then a mixture of fumaric acid (12.20 mg) and THF (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form W of the fumarate salt of the compound of formula (I). For the crystal form W, the XRPD pattern is shown in FIG. 36.

¹H NMR (400 MHz, DMSO-d₆) δ=11.02 (s, 1H), 7.53 (d, J=2.4 Hz, 1H), 7.44 (d, J=2.4 Hz, 1H), 7.40-7.34 (m, 1H), 7.24-7.18 (m, 1H), 7.01 (d, J=8.4 Hz, 1H), 6.82-6.77 (d, J=7.6 Hz, 1H), 6.47 (s, 1H), 4.16-4.06 (m, 1H), 3.97-3.90 (m, 2H), 3.43-3.37 (m, 2H), 2.78-2.70 (m, 4H), 2.61-2.55 (m, 6H), 2.34 (s, 3H), 2.23 (s, 3H), 1.91-1.83 (m, 2H), 1.71-1.52 (m, 10H), 1.18 (t, J=7.6 Hz, 3H)

### Example 25: Preparation of the Crystal Form X of the Fumarate Salt of the Compound of Formula (I)

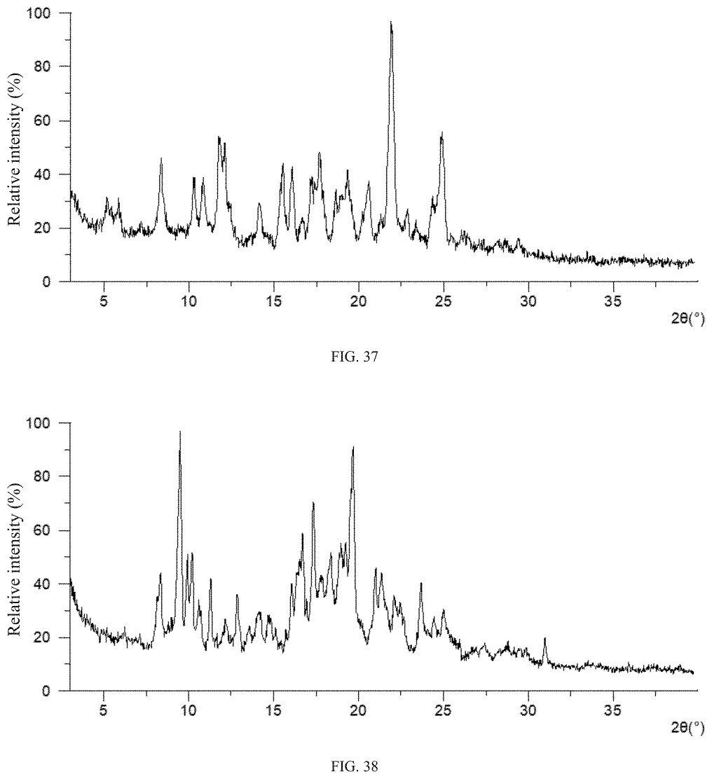

The crystal form A of the compound of formula (I) (100 mg) and acetonitrile (3 mL) were added to a reaction flask and the mixture was heated to 80° C. with stirring. Then a mixture of fumaric acid (12.76 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form X of the fumarate salt of the compound of formula (I). For the crystal form X, the XRPD pattern is shown in FIG. 37.

¹H NMR (400 MHz, DMSO-d₆) δ=11.02 (s, 1H), 7.55-7.50 (m, 1H), 7.44 (d, J=2.4 Hz, 1H), 7.37 (dd, J=2.4, 8.4 Hz, 1H), 7.22 (d, J=2.4 Hz, 1H), 7.00 (d, J=8.8 Hz, 1H), 6.80 (d, J=7.6 Hz, 1H), 6.49 (s, 1H), 4.15-4.08 (m, 1H), 3.94 (dd, J=2.8, 11.2 Hz, 2H), 3.43-3.39 (m, 2H), 2.77-2.72 (m, 4H), 2.61-2.53 (m, 6H), 2.33 (s, 3H), 2.24 (s, 3H), 1.87 (dd, J=2.0, 12.4 Hz, 2H), 1.70-1.51 (m, 10H), 1.19 (t, J=7.2 Hz, 3H)

### Example 26: Preparation of the Crystal Form Y of the Fumarate Salt of the Compound of Formula (I)

The crystal form A of the compound of formula (I) (100 mg) and THF (3 mL) were added to a reaction flask and the mixture was heated to 40° C. with stirring. Then a mixture of fumaric acid (12.44 mg) and THF (0.5 mL) was added and the mixture was stirred at 40° C. for another 60 hours. The above mixture was filtered and the filter cake was dried under vacuum at 50° C. to give the crystal form Y of the fumarate salt of the compound of formula (I). For the crystal form Y, the XRPD pattern is shown in FIG. 38.

¹H NMR (400 MHz, DMSO-d₆) δ=11.03 (s, 1H), 7.52 (d, J=2.8 Hz, 1H), 7.44 (d, J=2.4 Hz, 1H), 7.37 (dd, J=2.4, 8.8 Hz, 1H), 7.22 (d, J=2.4 Hz, 1H), 7.00 (d, J=8.8 Hz, 1H), 6.80 (d, J=7.6 Hz, 1H), 6.48 (s, 1H), 4.17-4.05 (m, 1H), 3.99-3.89 (m, 2H), 3.43-3.38 (m, 2H), 2.79-2.70 (m, 4H), 2.59-2.54 (m, 6H), 2.34 (s, 3H), 2.23 (s, 3H), 1.87 (dd, J=2.0, 12.4 Hz, 2H), 1.70-1.49 (m, 10H), 1.19 (t, J=7.2 Hz, 3H)

### Example 27: Preparation of the Crystal Form Z of the Fumarate Salt of the Compound of Formula (I)

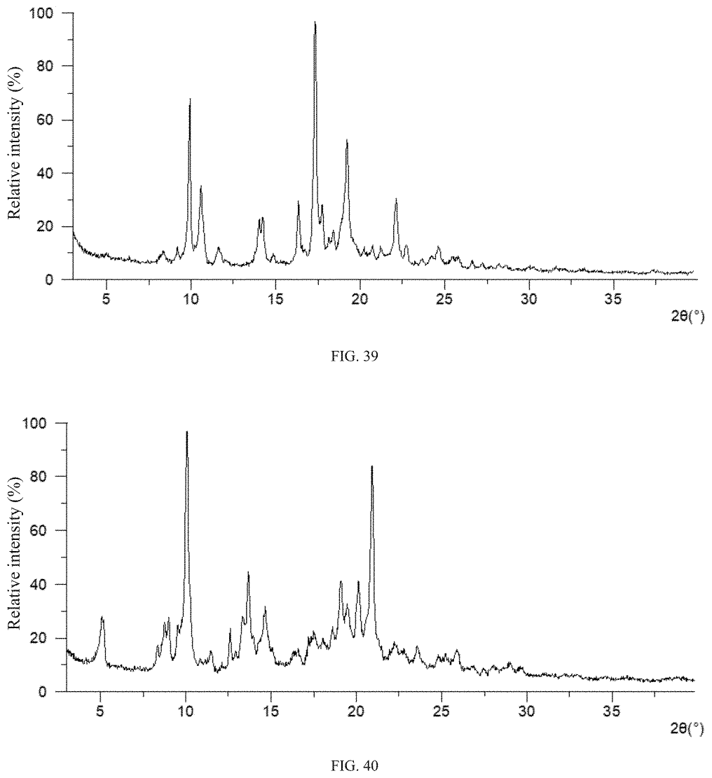

The crystal form A of the compound of formula (I) (100 mg) and THF (3 mL) were added to a reaction flask and the mixture was heated to 40° C. with stirring. Then a mixture of fumaric acid (12.44 mg) and THF (0.5 mL) was added and the mixture was stirred at 40° C. for another 60 hours. The above mixture was filtered and the filter cake was dried under vacuum at 80° C. to give the crystal form Z of the fumarate salt of the compound of formula (I). For the crystal form Z, the XRPD pattern is shown in FIG. 39.

¹H NMR (400 MHz, DMSO-d₆) δ=11.02 (s, 1H), 7.52 (d, J=2.4 Hz, 1H), 7.44 (d, J=2.4 Hz, 1H), 7.37 (dd, J=2.4, 8.4 Hz, 1H), 7.21 (d, J=2.8 Hz, 1H), 7.00 (d, J=8.4 Hz, 1H), 6.79 (d, J=7.6 Hz, 1H), 6.49 (s, 1H), 4.17-4.05 (m, 1H), 3.98-3.90 (m, 2H), 3.46-3.37 (m, 2H), 2.79-2.71 (m, 4H), 2.602.52 (m, 6H), 2.31 (s, 3H), 2.24 (s, 3H), 1.87 (dd, J=2.0, 12.4 Hz, 2H), 1.72-1.49 (m, 10H), 1.19 (t, J=7.2 Hz, 3H)

### Example 28: Preparation of the Crystal Form AA of the Fumarate Salt of the Compound of Formula (I)

The crystal form A of the compound of formula (I) (100 mg) and isopropyl alcohol (3 mL) were added to a reaction flask and the mixture was heated to 40° C. with stirring. Then a mixture of fumaric acid (12.71 mg) and isopropyl alcohol (0.5 mL) was added and the mixture was stirred at 40° C. for another 60 hours. The above mixture was filtered and the filter cake was dried under vacuum at 50° C. to give the crystal form AA of the fumarate salt of the compound of formula (I). For the crystal form AA, the XRPD pattern is shown in FIG. 40.

¹H NMR (400 MHz, DMSO-d₆) δ=11.03 (s, 1H), 7.52 (d, J=2.4 Hz, 1H), 7.44 (d, J=2.4 Hz, 1H), 7.37 (dd, J=2.4, 8.8 Hz, 1H), 7.22 (d, J=2.8 Hz, 1H), 7.00 (d, J=8.8 Hz, 1H), 6.80 (d, J=7.6 Hz, 1H), 6.48 (s, 1H), 4.17-4.05 (m, 1H), 3.94 (br dd, J=2.8, 11.2 Hz, 2H), 3.43-3.38 (m, 2H), 2.78-2.71 (m, 4H), 2.60-2.52 (m, 6H), 2.31 (s, 3H), 2.24 (s, 3H), 1.87 (dd, J=2.4, 12.4 Hz, 2H), 1.70-1.48 (m, 10H), 1.19 (t, J=7.2 Hz, 3H)

### Example 29: Preparation of the Crystal Form BB of the Fumarate Salt of the Compound of Formula (I)

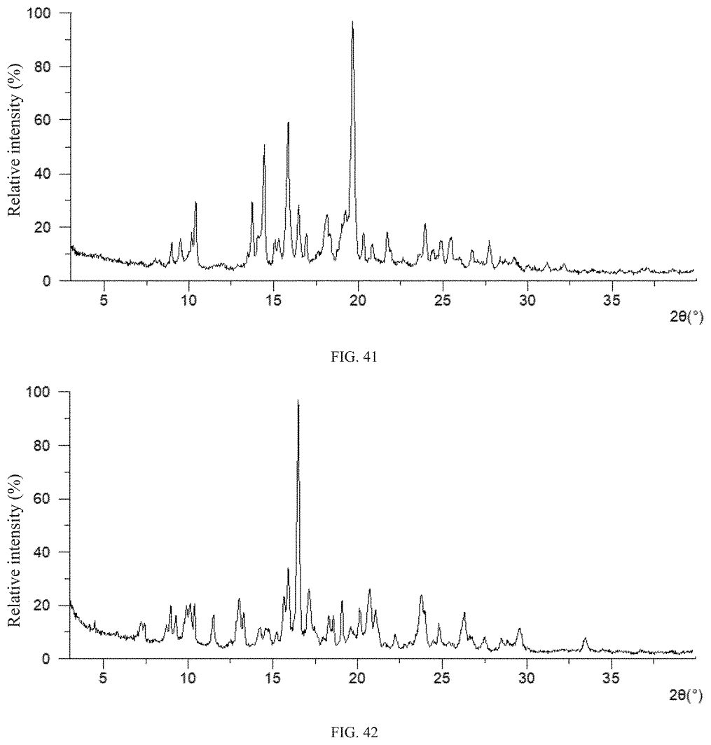

The crystal form A of the compound of formula (I) (100 mg) and acetonitrile (3 mL) were added to a reaction flask and the mixture was heated to 40° C. with stirring. Then a mixture of fumaric acid (12.06 mg) and acetonitrile (0.5 mL) was added and the mixture was stirred at 40° C. for another 60 hours. The above mixture was filtered and the filter cake was dried under vacuum at 50° C. to give the crystal form BB of the fumarate salt of the compound of formula (I). For the crystal form BB, the XRPD pattern is shown in FIG. 41.

¹H NMR (400 MHz, DMSO-d₆) δ=11.04 (s, 1H), 7.53 (d, J=2.8 Hz, 1H), 7.45 (d, J=2.4 Hz, 1H), 7.38 (dd, J=2.4, 8.8 Hz, 1H), 7.23 (d, J=2.8 Hz, 1H), 7.00 (d, J=8.8 Hz, 1H), 6.81 (d, J=7.6 Hz, 1H), 6.48 (s, 1H), 4.18-4.06 (m, 1H), 3.95 (dd, J=2.8, 11.2 Hz, 2H), 3.45-3.37 (m, 2H), 2.79-2.70 (m, 4H), 2.62-2.52 (m, 6H), 2.33 (s, 3H), 2.24 (s, 3H), 1.87 (dd, J=2.0, 12.4 Hz, 2H), 1.72-1.48 (m, 10H), 1.19 (t, J=7.2 Hz, 3H)

### Example 30: Preparation of the Crystal Form CC of the Fumarate Salt of the Compound of Formula (I)

The crystal form A of the compound of formula (I) (100 mg) and ethanol (3 mL) were added to a reaction flask and the mixture was heated to 40° C. with stirring. Then a mixture of fumaric acid (12.38 mg) and ethanol (0.5 mL) was added and the mixture was stirred at 40° C. for another 60 hours. The above mixture was filtered and the filter cake was dried under vacuum at 50° C. to give the crystal form CC of the fumarate salt of the compound of formula (I). For the crystal form CC, the XRPD pattern is shown in FIG. 42.

¹H NMR (400 MHz, DMSO-d₆) δ=11.03 (s, 1H), 7.52 (d, J=2.8 Hz, 1H), 7.44 (d, J=2.4 Hz, 1H), 7.37 (dd, J=2.4, 8.4 Hz, 1H), 7.22 (d, J=2.8 Hz, 1H), 6.99 (d, J=8.8 Hz, 1H), 6.80 (d, J=7.6 Hz, 1H), 6.48 (s, 1H), 4.17-4.04 (m, 1H), 3.94 (dd, J=3.2, 10.8 Hz, 2H), 3.44-3.37 (m, 2H), 2.79-2.70 (m, 4H), 2.62-2.53 (m, 6H), 2.32 (s, 3H), 2.23 (s, 3H), 1.91-1.80 (m, 2H), 1.72-1.48 (m, 10H), 1.18 (t, J=7.2 Hz, 3H)

### Example 31: Preparation of the Crystal Form DD of the Fumarate Salt of the Compound of Formula (I)

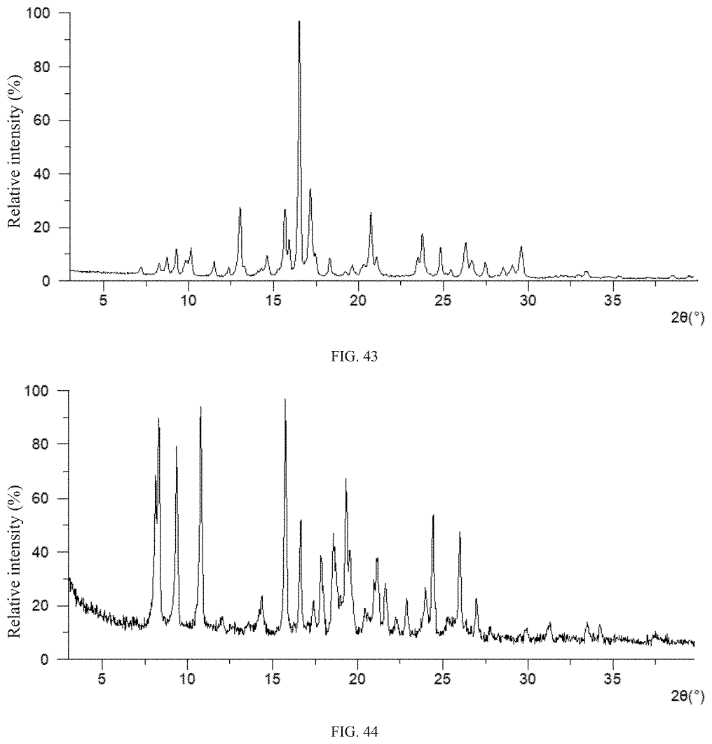

The crystal form A of the compound of formula (I) (100 mg) and water (3 mL) were added to a reaction flask and the mixture was heated to 40° C. with stirring. Then a mixture of fumaric acid (12.52 mg) and water (0.5 mL) was added and the mixture was stirred at 40° C. for another 60 hours. The above mixture was filtered and the filter cake was dried under vacuum at 50° C. to give the crystal form DD of the fumarate salt of the compound of formula (I). For the crystal form DD, the XRPD pattern is shown in FIG. 43.

¹H NMR (400 MHz, DMSO-d₆) δ=11.03 (s, 1H), 7.52 (d, J=2.4 Hz, 1H), 7.44 (d, J=2.4 Hz, 1H), 7.37 (dd, J=2.4, 8.8 Hz, 1H), 7.22 (d, J=2.8 Hz, 1H), 7.00 (d, J=8.8 Hz, 1H), 6.80 (d, J=7.2 Hz, 1H), 6.48 (s, 1H), 4.18-4.04 (m, 1H), 3.94 (dd, J=2.8, 11.2 Hz, 2H), 3.43-3.38 (m, 2H), 2.77-2.70 (m, 4H), 2.61-2.52 (m, 6H), 2.31 (s, 3H), 2.23 (s, 3H), 1.92-1.83 (m, 2H), 1.71-1.41 (m, 10H), 1.19 (t, J=7.2 Hz, 3H).

### Example 32: Preparation of the Crystal Form EE of the Fumarate Salt of the Compound of Formula (I)

The crystal form A of the compound of formula (I) (100 mg) and THF (2 mL) were added to a reaction flask and the mixture was heated to 80° C. with stirring. Then a mixture of fumaric acid (23.65 mg) and THF (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form EE of the fumarate salt of the compound of formula (I). For the crystal form EE, the XRPD pattern is shown in FIG. 44.

¹H NMR (400 MHz, DMSO-d₆) δ=11.02 (s, 1H), 7.52 (d, J=2.4 Hz, 1H), 7.44 (d, J=2.4 Hz, 1H), 7.37 (dd, J=2.4, 8.8 Hz, 1H), 7.21 (d, J=2.8 Hz, 1H), 7.00 (d, J=8.8 Hz, 1H), 6.80 (d, J=7.6 Hz, 1H), 6.51 (s, 2H), 4.16-4.05 (m, 1H), 3.96-3.92 (m, 2H), 3.43-3.37 (m, 2H), 2.74-2.67 (m, 8H), 2.58 (q, J=7.2 Hz, 2H), 2.43 (s, 3H), 2.24 (s, 3H), 1.88-1.85 (m, 2H), 1.70-1.54 (m, 10H), 1.19 (t, J=7.2 Hz, 3H)

### Example 33: Preparation of the Crystal Form FF of the Fumarate Salt of the Compound of Formula (I)

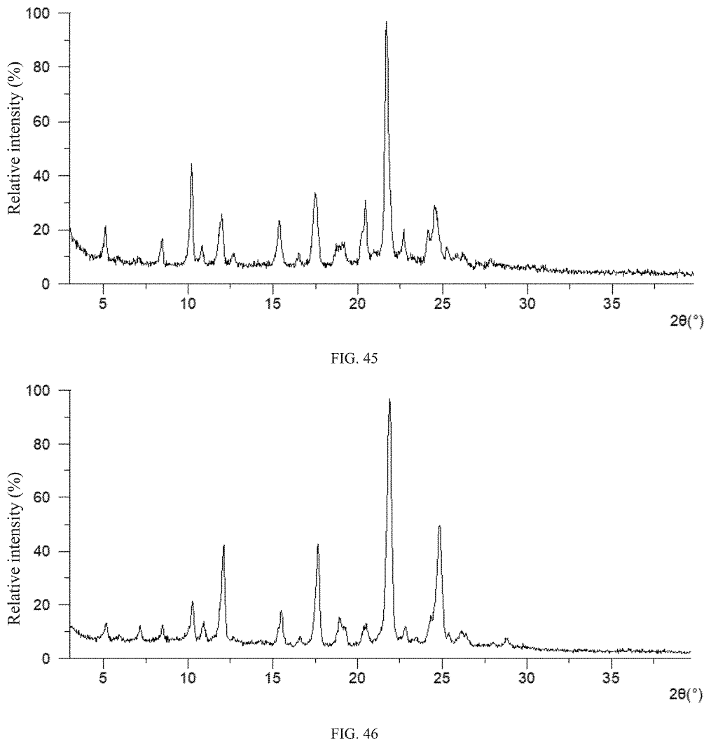

The crystal form A of the compound of formula (I) (100 mg) and isopropyl alcohol (3 mL) were added to a reaction flask and the mixture was heated to 80° C. with stirring. Then a mixture of fumaric acid (24.15 mg) and isopropyl alcohol (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form FF of the fumarate salt of the compound of formula (I). For the crystal form FF, the XRPD pattern is shown in FIG. 45.

¹H NMR (400 MHz, DMSO-d₆) δ=11.03 (s, 1H), 7.52 (d, J=2.8 Hz, 1H), 7.44 (d, J=2.4 Hz, 1H), 7.37 (dd, J=2.4, 8.8 Hz, 1H), 7.22 (d, J=2.4 Hz, 1H), 7.00 (d, J=8.8 Hz, 1H), 6.80 (d, J=7.6 Hz, 1H), 6.51 (s, 2H), 4.17-4.05 (m, 1H), 3.94 (dd, J=3.2, 11.2 Hz, 2H), 3.43-3.37 (m, 2H), 2.78-2.64 (m, 8H), 2.58 (q, J=7.2 Hz, 2H), 2.42 (s, 3H), 2.24 (s, 3H), 1.87 (dd, J=2.0, 12.4 Hz, 2H), 1.71-1.46 (m, 10H), 1.19 (t, J=7.2 Hz, 3H)

### Example 34: Preparation of the Crystal Form GG of the Fumarate Salt of the Compound of Formula (I)

The crystal form A of the compound of formula (I) (100 mg) and acetonitrile (3 mL) were added to a reaction flask and the mixture was heated to 80° C. with stirring. Then a mixture of fumaric acid (25.52 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form GG of the fumarate salt of the compound of formula (I). For the crystal form GG, the XRPD pattern is shown in FIG. 46.

¹H NMR (400 MHz, DMSO-d₆) δ=11.03 (s, 1H), 7.55-7.50 (m, 1H), 7.44 (d, J=2.4 Hz, 1H), 7.40-7.35 (m, 1H), 7.22 (s, 1H), 7.00 (d, J=8.8 Hz, 1H), 6.82-6.78 (m, 1H), 6.51 (s, 2H), 4.17-4.06 (m, 1H), 3.94 (dd, J=2.8, 11.2 Hz, 2H), 3.43-3.37 (m, 2H), 2.78-2.66 (m, 8H), 2.62-2.54 (m, 2H), 2.43 (s, 3H), 2.24 (s, 3H), 1.87 (dd, J=2.4, 12.4 Hz, 2H), 1.70-1.51 (m, 10H), 1.19 (t, J=7.2 Hz, 3H)

### Example 35: Preparation of the Crystal Form HH of the DL-Malate Salt of the Compound of Formula (I)

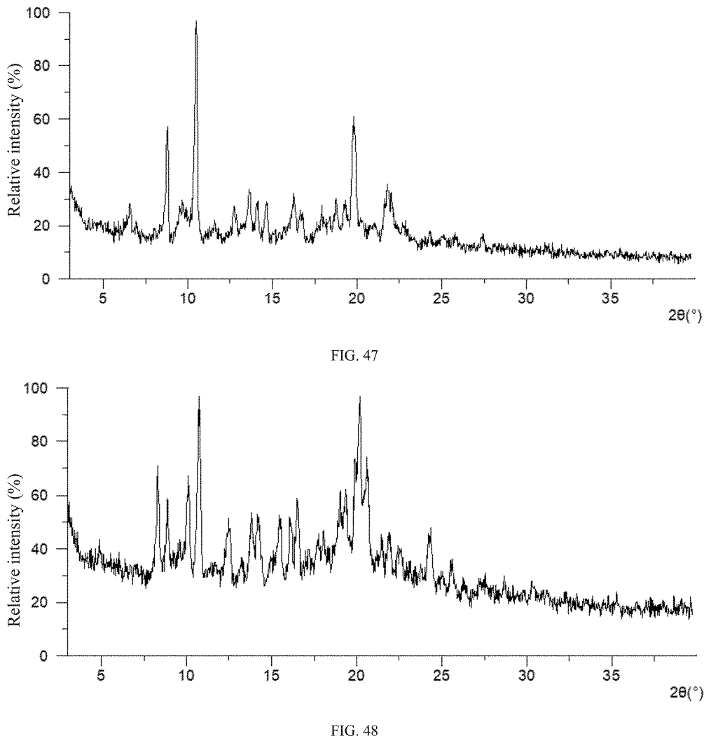

The crystal form A of the compound of formula (I) (100 mg) and isopropyl alcohol (3 mL) were added to a reaction flask and the mixture was heated to 80° C. with stirring. Then a mixture of DL-malic acid (15.53 mg) and isopropyl alcohol (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form HH of the DL-malate salt of the compound of formula (I). For the crystal form HH, the XRPD pattern is shown in FIG. 47.

¹H NMR (400 MHz, DMSO-d₆) δ=11.04 (s, 1H), 7.53 (d, J=2.4 Hz, 1H), 7.45 (d, J=2.4 Hz, 1H), 7.38 (dd, J=2.4, 8.8 Hz, 1H), 7.23 (d, J=2.4 Hz, 1H), 7.00 (d, J=8.8 Hz, 1H), 6.81 (d, J=7.6 Hz, 1H), 4.17-4.05 (m, 1H), 3.95 (dd, J=2.4, 11.2 Hz, 2H), 3.85 (dd, J=3.2, 10.4 Hz, 1H), 3.47-3.36 (m, 2H), 2.82-2.63 (m, 8H), 2.59 (q, J=7.2 Hz, 2H), 2.44 (s, 3H), 2.34-2.29 (m, 1H), 2.24 (s, 3H), 1.88 (dd, J=2.0, 12.4 Hz, 2H), 1.72-1.49 (m, 10H), 1.19 (t, J=7.2 Hz, 3H)

### Example 36: Preparation of the Crystal Form II of the DL-Malate Salt of the Compound of Formula (I)

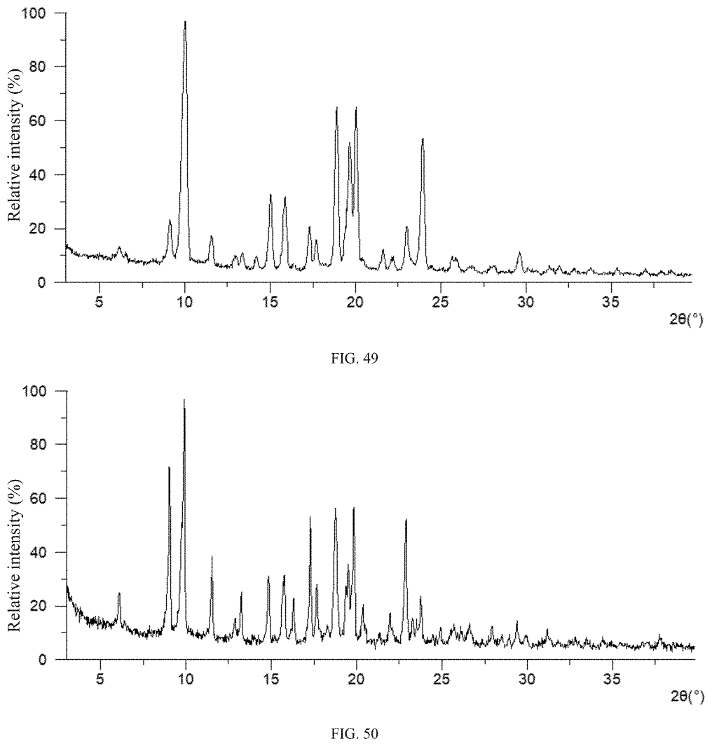

The crystal form A of the compound of formula (I) (100 mg) and acetonitrile (3 mL) were added to a reaction flask and the mixture was heated to 80° C. with stirring. Then a mixture of DL-malic acid (14.45 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form II of the DL-malate salt of the compound of formula (I). For the crystal form II, the XRPD pattern is shown in FIG. 48.

¹H NMR (400 MHz, DMSO-d₆) δ=11.03 (s, 1H), 7.53 (d, J=2.8 Hz, 1H), 7.44 (d, J=2.4 Hz, 1H), 7.38 (dd, J=2.4, 8.8 Hz, 1H), 7.22 (d, J=2.8 Hz, 1H), 7.00 (d, J=8.8 Hz, 1H), 6.80 (d, J=7.6 Hz, 1H), 4.17-4.05 (m, 1H), 3.94 (dd, J=3.2, 11.2 Hz, 2H), 3.85 (dd, J=3.6, 10.4 Hz, 1H), 3.46-3.36 (m, 2H), 2.80-2.68 (m, 8H), 2.58 (q, J=7.4 Hz, 2H), 2.48-2.44 (m, 3H), 2.34-2.27 (m, 1H), 2.22 (s, 3H), 1.87 (dd, J=2.0, 12.4 Hz, 2H), 1.73-1.51 (m, 10H), 1.19 (t, J=7.2 Hz, 3H)

### Example 37: Preparation of the Crystal Form JJ of the DL-Malate Salt of the Compound of Formula (I)

The crystal form A of the compound of formula (I) (100 mg) and acetonitrile (3 mL) were added to a reaction flask and the mixture was heated to 80° C. with stirring. Then a mixture of DL-malic acid (28.51 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form JJ of the DL-malate salt of the compound of formula (I). For the crystal form JJ, the XRPD pattern is shown in FIG. 49.

¹H NMR (400 MHz, DMSO-d₆) δ=11.04 (s, 1H), 7.53 (d, J=2.8 Hz, 1H), 7.45 (d, J=2.4 Hz, 1H), 7.38 (dd, J=2.4, 8.4 Hz, 1H), 7.22 (d, J=2.8 Hz, 1H), 7.00 (d, J=8.8 Hz, 1H), 6.81 (d, J=7.6 Hz, 1H), 4.17-4.05 (m, 1H), 3.99-3.88 (m, 3H), 3.43-3.36 (m, 2H), 3.00-2.89 (m, 4H), 2.76-2.74 (m, 4H), 2.65-2.54 (m, 5H), 2.36-2.30 (m, 1H), 2.24 (s, 3H), 1.91-1.83 (m, 2H), 1.72-1.55 (m, 10H), 1.19 (t, J=7.2 Hz, 3H)

### Example 38: Preparation of the Crystal Form KK of the L-Malate Salt of the Compound of Formula (I)

The crystal form A of the compound of formula (I) (100 mg) and acetonitrile (3 mL) were added to a reaction flask and the mixture was heated to 80° C. with stirring. Then a mixture of L-malic acid (27.01 mg) and acetonitrile (0.5 mL) was added and the mixture was stirred at 80° C. for another 1 hour. The heating was turned off, and the mixture was allowed to naturally cool down to room temperature and stirred for 12 hours. The above mixture was filtered and the filter cake was dried under vacuum at 50° C. for 12 hours to give the crystal form KK of the L-malate salt of the compound of formula (I). For the crystal form KK, the XRPD pattern is shown in FIG. 50.

¹H NMR (400 MHz, DMSO-d₆) δ=11.04 (s, 1H), 7.54 (d, J=2.8 Hz, 1H), 7.46 (d, J=2.4 Hz, 1H), 7.39 (dd, J=2.4, 8.6 Hz, 1H), 7.23 (d, J=2.4 Hz, 1H), 7.01 (d, J=8.8 Hz, 1H), 6.81 (d, J=7.6 Hz, 1H), 4.17-4.04 (m, 1H), 4.00-3.88 (m, 3H), 3.43-3.38 (m, 2H), 3.01-2.87 (m, 4H), 2.80-2.71 (m, 4H), 2.66-2.55 (m, 5H), 2.38-2.30 (m, 1H), 2.25 (s, 3H), 1.88 (dd, J=2.0, 12.4 Hz, 2H), 1.74-1.56 (m, 10H), 1.19 (t, J=7.2 Hz, 3H)

### Example 39: Preparation of the Crystal Form LL of the L-Malate Salt of the Compound of Formula (I)

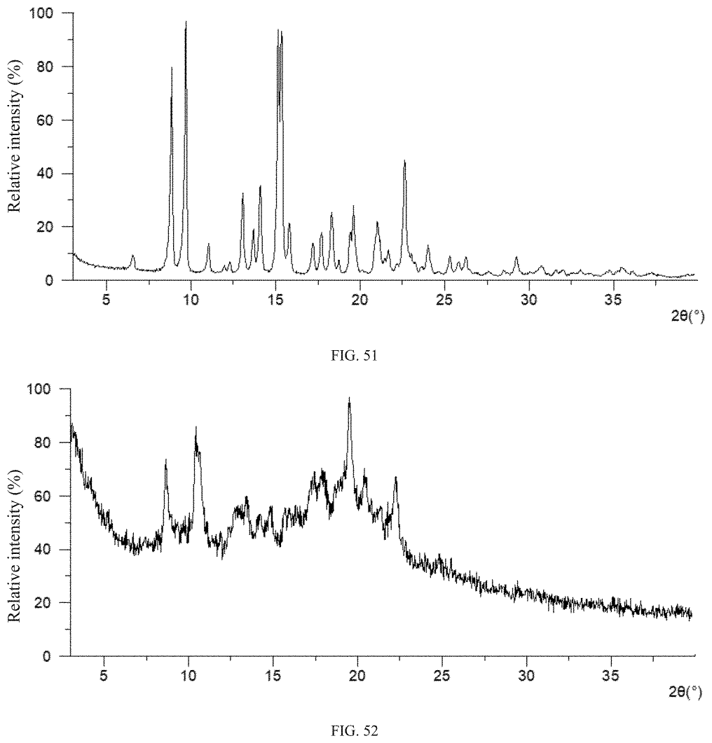

The crystal form A of the compound of formula (I) (300.16 mg) and acetonitrile (3 mL) were added to a reaction flask and the mixture was heated to 80° C. with stirring. Then a mixture of L-malic acid (81.46 mg) and acetonitrile (0.5 mL) was added and the mixture was stirred at 80° C. for another 1 hour. The heating was turned off, and the mixture was allowed to naturally cool down to room temperature and stirred for 72 hours. The above mixture was filtered and the filter cake was dried under vacuum at 50° C. to give the crystal form LL of the L-malate salt of the compound of formula (I). For the crystal form LL, the XRPD pattern is shown in FIG. 51.

¹H NMR (400 MHz, DMSO-d₆) δ=11.04 (s, 1H), 7.54 (d, J=2.8 Hz, 1H), 7.45 (d, J=2.4 Hz, 1H), 7.38 (dd, J=2.4, 8.4 Hz, 1H), 7.24 (d, J=2.4 Hz, 1H), 7.00 (d, J=8.8 Hz, 1H), 6.82 (d, J=7.6 Hz, 1H), 4.17-4.05 (m, 1H), 4.00-3.86 (m, 3H), 3.423.38 (m, 2H), 2.96-2.90 (m, 4H), 2.81-2.71 (m, 4H), 2.65-2.54 (m, 5H), 2.37-2.29 (m, 1H), 2.24 (s, 3H), 1.87 (dd, J=2.0, 12.4 Hz, 2H), 1.73-1.55 (m, 10H), 1.18 (t, J=7.2 Hz, 3H)

### Example 40: Preparation of the Crystal Form MM of the L-Malate Salt of the Compound of Formula (I)

The crystal form A of the compound of formula (I) (12 g) and isopropyl alcohol (100 mL) were added to a reaction flask and the mixture was heated to 80° C. with stirring. Then L-malic acid (2.99 g) was added and the mixture was stirred at 80° C. for another 0.5 hours. The heating was turned off, and the mixture was allowed to naturally cool down to room temperature and stirred for 12 hours. The above mixture was filtered. The filter cake was washed with isopropyl alcohol (30 mL), and then dried under vacuum at 50° C. to give the crystal form MM of the L-malate salt of the compound of formula (I). For the crystal form MM, the XRPD pattern is shown in FIG. 52.

¹H NMR (400 MHz, DMSO-d₆) δ=11.03 (s, 1H), 7.53 (d, J=2.4 Hz, 1H), 7.45 (d, J=2.4 Hz, 1H), 7.38 (dd, J=2.4, 8.8 Hz, 1H), 7.21 (d, J=2.4 Hz, 1H), 7.00 (d, J=8.8 Hz, 1H), 6.81 (d, J=7.6 Hz, 1H), 4.17-4.03 (m, 1H), 3.99-3.88 (m, 3H), 3.45-3.35 (m, 2H), 2.98-2.90 (m, 4H), 2.79-2.72 (m, 4H), 2.66-2.54 (m, 5H), 2.35-2.30 (m, 1H), 2.24 (s, 3H), 1.87 (dd, J=2.0, 12.8 Hz, 2H), 1.71-1.55 (m, 10H), 1.18 (t, J=7.2 Hz, 3H)

### Example 41: Preparation of the Crystal Form NN of the L-Malate Salt of the Compound of Formula (I)

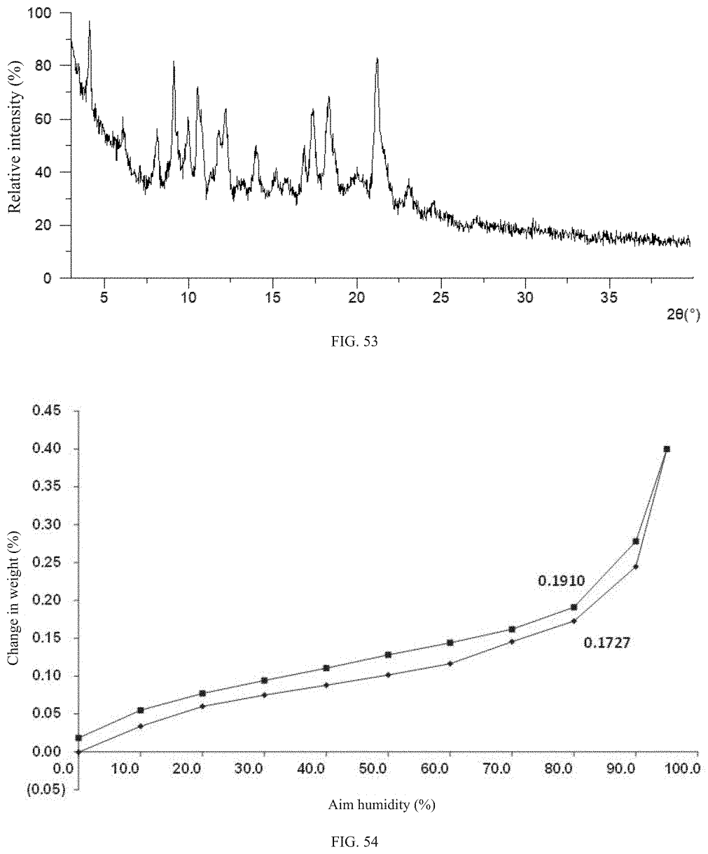

The crystal form A of the compound of formula (I) (0.3 g) and isopropyl alcohol (6 mL) were added to a reaction flask and the mixture was heated to 80° C. with stirring. Then L-malic acid (74.73 g) was added and the mixture was stirred at 80° C. for another 0.5 hours. The heating was turned off, and the mixture was allowed to naturally cool down to room temperature and stirred for 24 hours. The above mixture was filtered. The filter cake was washed with ethanol (30 mL), and then dried under vacuum to give the crystal form NN of the L-malate salt of the compound of formula (I). For the crystal form NN, the XRPD pattern is shown in FIG. 53.

¹H NMR (400 MHz, DMSO-d₆) δ=11.03 (s, 1H), 7.53 (d, J=2.4 Hz, 1H), 7.45 (d, J=2.4 Hz, 1H), 7.38 (dd, J=2.4, 8.4 Hz, 1H), 7.22 (d, J=2.4 Hz, 1H), 7.00 (d, J=8.8 Hz, 1H), 6.80 (d, J=7.6 Hz, 1H), 4.17-4.04 (m, 1H), 3.99-3.85 (m, 3H), 3.43-3.37 (m, 4H), 2.92-2.85 (m, 4H), 2.80-2.71 (m, 4H), 2.62-2.54 (m, 5H), 2.36-2.29 (m, 1H), 2.24 (s, 3H), 1.91-1.83 (m, 2H), 1.72-1.53 (m, 10H), 1.19 (t, J=7.2 Hz, 3H)

### Assay Example 42: Stability Assay of the Crystal Form C of the Compound of Formula (I)

Assay Procedure:

1. No. 1-5: The crystal form C of the compound of formula (I) (200 mg) and the solvent (4 mL) were added to a reaction flask, and the mixture was heated to 50° C. and stirred for 48 h. Then the above mixture was filtered and the filter cake was dried under vacuum at 60° C. to obtain a solid, which was detected by XRPD.
2. No. 6-8: The crystal form C of the compound of formula (I) (200 mg) and the solvent (4 mL) were added to a reaction flask, and the mixture was kept at 25° C. and stirred for 7 days. Then the above mixture was filtered and the filter cake was dried under vacuum at 60° C. to obtain a solid, which was detected by XRPD.
3. No. 9: The powder of the crystal form C of the compound of formula (I) was added to a circular mold (6 mm diameter). The powder was pressurized until the pressure reached about 350 MPa, and then the pressed sample was directly spread on a XRPD disk for testing.

Assay Results:

| | | Crystal |
|---|---|---|
| No. | Solvent | form |
| 1 | acetonitrile | C |
| 2 | Acetone | C |
| 3 | n-Butanol | C |
| 4 | Butanone | C |
| 5 | Water | C |
| 6 | 1,4-Dioxane | C |
| 7 | 1,4-Dioxane: water (1:1) | C |
| 8 | Ethanokwater (7:3) | C |
| 9 | — | C |

Assay Conclusion:

The compound of the present disclosure (i.e., the crystal form C of the compound of formula (I)) has a stable crystal form in a variety of solvents or under a tablet-pressing condition.

### Assay Example 43: Assay of Solubility

Assay method: The crystal form C of the compound of formula (I) was weighed and added to a 4 mL glass vial, and then 2 mL of solvent was added. The mixture was mixed well and a magnet bar was added to the above suspension. The suspension was stirred on a heater with magnetic stirring (37° C., protected from light). After 24 hours of stirring, a sample was taken. The resulting sample solution was quickly centrifuged. The supernatant was diluted to the appropriate multiple, and the concentration was determined by HPLC (unit: mg/mL).

Assay results: See Table 7.

Table 7. Results of solubility assay

TABLE 7
Results of solubility assay

| | Assay compound | | | | | | |
|---|---|---|---|---|---|---|---|
| | Buffer solution | | | | Biological medium | | |
| | pH 1.0 | pH 3.8 | pH 4.5 | pH 5.5 | FaSSIF | FeSSIF | SGF |
| The crystal form C of | >10 | >10 | >10 | 0.81 | 0.11 | 1.11 | 1.49 |
| the compound of | | | | | | | |
| formula (I) | | | | | | | |

Assay conclusion: The crystal form C of the compound of formula (I) has ideal solubility in different acidic vehicles.

### Assay Example 1: Assay of FLT3 Inhibitory Activity In Vitro

Assay Materials:

FLT3 Kinase Enzyme System (kinase system) was purchased from Promega. Envision Multi-Label Analyzer (PerkinElmer).

Assay Method:

The enzyme, substrate, ATP (adenosine triphosphate) and inhibitors were diluted using the buffer solution in the kit.

The compounds to be tested were 5-fold serially diluted with a pipette to obtain eight concentrations, i.e., from 5 μmol/L to 0.065 nmol/L, and the final concentration of dimethyl sulfoxide was 5%. The assay was carried out in duplicate. 1 μl of the inhibitors of each concentration gradient, 2 μl of the FLT3 enzyme (15 ng), and a 2 μl mixture of the substrate and ATP (50 μmol/L ATP, 0.1 μg/μL MBP) were added to the microplate. At this time, the final concentration gradient of the compounds was from 1 μmol/L to 0.013 nmol/L. The system was reacted at 30° C. for 120 minutes. After the reaction was completed, 5 μL of ADP-Glo reagent was added to each well. The system was further reacted at 30° C. for 40 minutes. After the reaction was completed, 10 μL of kinase detection reagent was added to each well and reacted at 30° C. for 30 minutes. The chemiluminescence was then read by the PerkinElmer Envision multi-label analyzer with an integration time of 0.5 seconds.

Data Analysis:

The original data was converted into an inhibition rate, and the IC₅₀ value can be obtained by four-parameter curve fitting. The inhibitory activity of the compound of the present disclosure on FLT3 enzyme was provided in Table 8.

Assay Results: See Table 8.

Conclusion: The compound of the present disclosure has excellent in vitro inhibitory activity against FLT3.

TABLE 8

| Sample | FLT3 IC₅₀ (nmol/L) |
|---|---|
| Trifluoroacetate salt of compound A | 4.02 |
| Compound B | 0.81 |
| Trifluoroacetate salt of | 0.42 |
| the compound of formula (I) | |

### Assay Example 2: Assay of AXL Inhibitory Activity In Vitro

Assay Materials:

AXL Kinase Enzyme System (kinase system) was purchased from Promega. Envision multi-label Analyzer (PerkinElmer).

Assay Method:

The enzyme, substrate, ATP and inhibitors were diluted using the buffer solution in the kit.

The compounds to be tested were 5-fold serially diluted with a pipette to obtain eight concentrations, i.e., from 5 μmol/L to 0.065 nmol/L, and the final concentration of dimethyl sulfoxide was 5%. The assay was carried out in duplicate. 1 μl of the inhibitors of each concentration gradient, 2 μl of the AXL enzyme (6 ng), and a 2 μl mixture of the substrate and ATP (50 μmol/L ATP, 0.2 μg/μL Axltide) were added to the microplate. At this time, the final concentration gradient of the compounds was from 1 μmol/L to 0.013 nmol/L. The system was reacted at 30° C. for 60 minutes. After the reaction was completed, 5 μL of ADP-Glo reagent was added to each well. The system was further reacted at 30° C. for 40 minutes. After the reaction was completed, 10 μL of kinase detection reagent was added to each well and reacted at 30° C. for 30 minutes. The chemiluminescence was then read by the PerkinElmer Envision multi-label analyzer with an integration time of 0.5 seconds.

Data Analysis:

The original data was converted into an inhibition rate, and the IC₅₀ value can be obtained by four-parameter curve fitting. The inhibitory activity of the compound of the present disclosure on AXL enzyme was provided in Table 9.

Assay Results: See Table 9.

Conclusion: The compound of the present disclosure has excellent in vitro inhibitory activity against AXL.

TABLE 9

| Sample | AXL IC₅₀ (nmol/L) |
|---|---|
| Trifluoroacetate salt of compound A | 5.76 |
| Compound B | 1.37 |
| Trifluoroacetate salt of | 1.22 |
| the compound of formula (I) | |

### Assay Example 3: Assay of Inhibiting Proliferation of FLT3 Mutant In Vitro

Assay Method:

The assay was carried out using KINOMEscan™ technology. The assay compounds were stored in 100% DMSO. The assay was carried out by means of 3-fold dilution and 11-point fitting. All compounds used for Kd determination were dispersed by ultrasound, then directly diluted and tested. All reactions were carried out in a 384-well polypropylene plate. Each portion has a final volume of 0.02 ml. The plate was incubated with shaking at room temperature for 1 hour, and then processed. The kinase concentration in the eluent was finally determined by qPCR method. Kd was obtained by fitting.

Assay Results: See Table 10.

TABLE 10

| | Kd (nmol/L) | |
|---|---|---|
| | | Trifluoroacetate salt |
| | | of the compound of |
| Target | Compound B | formula (I) |
| FLT3(D835H) | 3.2 | 2.3 |
| FLT3(D835V) | 0.53 | 0.23 |
| FLT3(D835Y) | 1.7 | 1.4 |
| FLT3(ITD) | 5.3 | 3.6 |
| FLT3(ITD, D835V) | 0.41 | 0.2 |
| FLT3(ITD, F691L) | 0.76 | 0.21 |
| FLT3(K663Q) | 59 | 10 |
| FLT3(N841I) | 8.2 | 5.4 |
| FLT3(R834Q) | 65 | 31 |

Conclusion: The compound of the present disclosure has excellent in vitro inhibitory activity against mutant FLT3 targets, which shows higher activity for all 10 mutants than that of the known compound B, wherein the activity for FLT3 (ITD, F691L) is 3.6 times higher and the activity for FLT3 (K663Q) is 5.9 times higher. Such higher activity against mutant FLT3 is of high clinical significance considering that the point mutation is an important cause of drug resistance of FLT3 inhibitors.

### Assay Example 4: Assay of Inhibiting Proliferation of MV-4-11 In Vitro

Assay Materials:

IMDM medium, fetal bovine serum, and penicillin/streptomycin antibiotics were purchased from Promega (Madison, Wis.). The MV-4-11 cell line was purchased from the Cell Bank of the Chinese Academy of Sciences. Envision Multi-Label Analyzer (PerkinElmer).

Assay Method:

MV-4-11 cells were seeded in a white 96-well plate with 80 μL of cell suspension per well containing 10,000 MV-4-11 cells. The cell plate was cultured overnight in a carbon dioxide incubator.

The compounds to be tested were 5-fold serially diluted with a pipette to obtain eight concentrations, i.e., from 2 mmol/L to 26 nmol/L. The assay was carried out in duplicate. 78 μL of medium was added to the intermediate plate, and then 2 μL per well of the serially diluted compounds were transferred to the intermediate plate according to the corresponding position. After mixing, an amount of 20 μL per well was transferred to the cell plate. The cell plate was cultured in a carbon dioxide incubator for 3 days.

25 μL per well of Promega CellTiter-Glo reagent was added to the cell plate and incubated at room temperature for 10 minutes to stabilize the luminescence signal. A PerkinElmer Envision multi-label analyzer was used for reading.

Data Analysis:

The original data was converted into an inhibition rate, and the IC₅₀ value can be obtained by four-parameter curve fitting. The inhibitory activity of the compound of the present disclosure on the proliferation of MV-4-11 cells was provided in Table 11.

Assay Results: See Table 11.

Conclusion: The compound of the present disclosure has excellent inhibitory activity on the proliferation of MV-4-11 cells.

TABLE 11

| Sample | MV-4-11 IC₅₀ (nmol/L) |
|---|---|
| Compound A | 5.4 |
| Compound B | 4.65 |
| Trifluoroacetate salt of | 3.02 |
| the compound of formula (I) | |

### Assay Example 5: In Vivo Pharmacokinetic Assay in Mice

Assay Purpose:

The purpose of this assay is to evaluate the pharmacokinetic behavior of the compound after single administration by intravenous injection and intragastric gavage, and to investigate the bioavailability after administration by intragastric gavage.

Assay Procedure:

CD-1 male mice aged 7 to 10 weeks were selected. The doses for intravenous and oral administration were 1 mg/kg and 2.5 mg/kg, respectively. The mice were fasted for at least 12 hours before the administration, and resumed feeding 4 hours after the administration. The mice had free access to water throughout the assay period.

On the day of the assay, the animals in the intravenous group were administrated the corresponding compound through a single injection in the tail vein at an administration volume of 5 mL/kg; the oral group were administrated the corresponding compound through a single intragastric gavage at an administration volume of 10 mL/kg. The animals were weighed before administration, and the administration volume was calculated based on the body weight. The time for collecting samples was: 0.083 (the injection group), 0.25, 0.5, 1, 2, 4, 8, 24 h. About 30 μL of whole blood was collected from the saphenous vein at each time point to prepare plasma for concentration determination by high-performance liquid chromatography-tandem mass spectrometry (LC-MS/MS). All animals were euthanized by CO₂ anesthesia after collecting the PK samples at the last time point. The plasma concentrations were processed by non-compartmental model of WinNonlin™ Version 6.3 (Pharsight, Mountain View, Calif.) pharmacokinetic software, and the pharmacokinetic parameters were calculated by the linear logarithm trapezoidal method.

Assay Results:

The results of in vivo PK property evaluation in mice are shown in Table 12.

Assay Conclusion:

The compound of the present disclosure has an appropriate clearance rate, has relatively good oral AUC and bioavailability, and has good pharmacokinetic properties in mice. The compound of the present disclosure has unexpectedly improved PK properties compared to compound A.

TABLE 12
Results of evaluation of in vivo pharmacokinetic properties

| | | | | Trifluoroacetate |
|---|---|---|---|---|
| | | Compound | Compound | salt of the compound |
| Administration | Parameter | A | B | of formula (I) |
| Injection | T_{1/2} (hr) | 2.13 | 2.09 | 2.40 |
| (1 mg/kg) | Vdss (L/Kg) | 8.93 | 7.47 | 8.63 |
| | Cl | 73.4 | 46.7 | 48.5 |
| | (mL/min/Kg) | | | |
| | AUC₀₋ₗₐₛₜ | 403 | 605 | 604 |
| | (nm · h) | | | |
| Oral | Cₘₐₓ (nM) | 13.4 | 85.3 | 60.5 |
| administration | Tₘₐₓ (h) | 1.00 | 1.00 | 4.00 |
| (2.5 mg/kg) | T_{1/2} (h) | 7.57 | 5.59 | 4.41 |
| | AUC₀₋ₗₐₛₜ | 74.5 | 501 | 598 |
| | (nM/hr) | | | |
| | F % | 7.39 | 33.1 | 37.3 |

### Assay Example 6: Assay of Inhibition of MV4-11 Subcutaneous Xenograft Tumor In Vivo

Assay Purpose:

In this assay, the anti-tumor effect of the compounds was evaluated using a nude mice model of subcutaneous xenograft tumor of human biphenotypic B bone marrow mononuclear leukemia cell MV4-11.

Assay Procedure:

Human biphenotypic B bone marrow mononuclear leukemia cell MV4-11 was cultured in vitro in suspension with a RPMI1640 medium supplemented with 10% fetal bovine serum, 100 U/mL penicillin and 100 μg/mL streptomycin in a 5% CO₂ cell incubator at 37° C. Routine passaging was performed twice a week. Cells in the logarithmic growth phase were collected, counted and then used for inoculation.

0.2 mL (1×10⁷) of MV4-11 cells (plus matrigel, with a volume ratio of 1:1) were inoculated subcutaneously on the right back of each mouse. The grouping and dosing were started when the average tumor volume reached about 140-200 mm³. The grouping and dosing regimen of the assay was shown in the table below.

The diameter of the tumor was measured with vernier calipers twice a week. The formula for calculating the tumor volume is: V=0.5a×b², where a and b represent the long and short diameters of the tumor, respectively.

Assay results: The tumor inhibitory effect of the compounds is shown in Table 13.

TABLE 13
Results of MV4-11 ectopic xenograft assay

| | | Compound | | | | |
|---|---|---|---|---|---|---|
| | | | | Trifluoroacetate salt of the | | |
| | | Blank | Compound B | compound of formula (I) | | |
| Dose (mg/kg) | | 0 | 1.5 | 1 | 1.5 | 4.5 |
| Dosing frequency | | Once a day | | | | |
| Route of | | Oral | | | | |
| administration | | | | | | |
| Average | Day 0 | 149 | | | | |
| tumor | Day 14 | 815 | 431 | 375 | 273 | 51 |
| volume | | | | | | |
| (mm³) | | | | | | |

| | | | | | |
|---|---|---|---|---|---|
| % Inhibition rate | — | 57.7 | 66.1 | 81.0 | 114.7 |
| on day 14 | | | | | |

Assay Conclusion:

The compound of the present disclosure has a significant inhibitory effect on the growth of xenograft tumor of human biphenotypic B bone marrow mononuclear leukemia cell MV4-11. A low dose (1 mg/kg) of the compound of the present disclosure shows better tumor inhibitory effect than that of a high dose (1.5 mg/kg) of compound B. At the same dose, the compound of the present disclosure has more significant advantage than compound B. A dose of 4.5 mpk can shrink a tumor.

### Assay Example 7: In Vivo Pharmacokinetic Assay in Rats

Assay Purpose:

The purpose of this assay is to evaluate the pharmacokinetic behavior of the compound after single administration by intravenous injection and intragastric gavage, and to investigate the bioavailability after administration by intragastric gavage.

Assay Procedure:

SD male rats aged 7 to 10 weeks were selected. The rats were fasted for at least 12 hours before the administration, and resumed feeding 4 hours after the administration. The rats had free access to water throughout the assay period.

On the day of the assay, the animals in the intravenous group were administrated the corresponding compound through a single injection in the tail vein at an administration volume of 5 mL/kg; the oral group were administrated the corresponding compound through a single intragastric gavage at an administration volume of 10 mL/kg. The animals were weighed before administration, and the administration volume was calculated based on the body weight. The time for collecting samples was: 0.083 (the injection group), 0.25, 0.5, 1, 2, 4, 6, 8, 24 h. About 200 μL of whole blood was collected from the jugular vein at each time point to prepare plasma for concentration determination by high-performance liquid chromatography-tandem mass spectrometry (LC-MS/MS). All animals were euthanized by CO₂ anesthesia after collecting the PK samples at the last time point. The plasma concentrations were processed by non-compartmental model of WinNonlin™ Version 6.3 (Pharsight, Mountain View, Calif.) pharmacokinetic software, and the pharmacokinetic parameters were calculated by the linear logarithm trapezoidal method.

Assay results: The results of in vivo PK property evaluation in rats are shown in Table 14.

TABLE 14
Results of evaluation of in vivo pharmacokinetic properties in rats

| | | | Trifluoroacetate | The crystal |
|---|---|---|---|---|
| | | | salt of the | form C of the |
| | | Compound | compound of | compound of |
| Administration | Parameter | B | formula (I) | formula (I) |
| Injection | Dose (mg/kg) | 1 | 1 | / |
| | T_{1/2} (hr) | 2.73 | 2.99 | |
| | Vdss (L/Kg) | 10.2 | 13.3 | |
| | Cl | 44.0 | 61.2 | |
| | (mL/min/Kg) | | | |
| | AUC₀₋ₗₐₛₜ | 587 | 457 | |
| | (nm · h) | | | |
| Oral | Dose (mg/kg) | 2.5 | 2.5 | 20 |
| | Cₘₐₓ (nM) | 29.1 | 40.6 | 483 |
| | Tₘₐₓ (h) | 6.00 | 8.00 | 7.00 |
| | AUC₀₋ₗₐₛₜ | 361 | 510 | 8457 |
| | (nM/hr) | | | |
| | F% | 24.6 | 44.7 | 93 |

Assay Conclusion:

The compound of the present disclosure has excellent oral AUC and bioavailability, and has good pharmacokinetic properties in rats. The compound of the present disclosure has unexpectedly improved PK properties compared to compound B. The crystal form C of the compound shows further improved bioavailability.

### Assay Example 8: Assay of Inhibition of Molm-13 Subcutaneous Xenograft Tumor In Vivo

Assay Purpose:

The purpose of this assay is to evaluate the pharmacodynamics of the compounds in the NOD/SCID female mice model of human acute myeloma MOLM-13 cell line subcutaneous xenograft.

Assay Procedure:

MOLM-13 cells were cultured in RPMI-1640 medium containing 10% fetal bovine serum. The MOLM-13 cells were collected in the exponential growth phase, and resuspended in PBS to a suitable concentration for subcutaneous tumor inoculation in nude mice.

The assay mice were inoculated with 5×10⁶ MOLM-13 cells resuspended in 0.1 ml of PBS (0.1 ml per mouse) subcutaneously on the right back. Tumor growth was regularly observed. When the tumor grew to an average volume of 98 mm³, the mice were randomly divided into groups and administered according to tumor size and body weight of the mice.

After the start of administration, the body weight and tumor size of the mice were measured three times a week. The formula for calculating the tumor volume is: tumor volume (mm³)=½×(a×b²) (where a represents the long diameter and b represents the short diameter).

Assay Results: The tumor inhibitory effect of the compounds is shown in Table 15.

Assay Conclusion:

The compound of the present disclosure has a significant inhibitory effect on the growth of human-derived Molm-13 xenograft tumors. The compound of the present disclosure shows better tumor inhibitory effect than that of compound B at the same dose (15 mg/kg). The tumor volume is reduced to zero at a dose of 50 mg/kg.

TABLE 15
Results of Molm-13 ectopic xenograft assay

| | | | | Compound | Compound |
|---|---|---|---|---|---|
| | | | Compound | of formula | of formula |
| Assay compound | | Blank | B | (I) | (I) |
| Dose (mg/kg) | | 0 | 15 | 15 | 50 |
| Dosing frequency | | Once a | Once a day | Once a day | Once a day |
| | | day | | | |
| Route of administration | | Oral | Oral | Oral | Oral |
| Average | Day 0 | 98.03 | 98.02 | 98.34 | 98.05 |
| tumor volume | Day 11 | 837.59 | 401.07 | 156.85 | 53.08 |
| (mm³) | Day 15 | 2322.0 | 566.95 | 223.09 | 0 |
| | | 8 | | | |

| | | | | |
|---|---|---|---|---|
| % Inhibition rate on day 11 | — | 59 | 92 | 106 |
| % Inhibition rate on day 15 | — | 79 | 94 | 104 |

### Assay Example 9: In Vivo Pharmacokinetic Assay in Dogs

Assay Purpose:

The purpose of this assay is to evaluate the pharmacokinetic behavior of the compound after single intravenous and gavage administration, and to investigate the bioavailability of the compound after gavage administration.

Assay Procedure:

Male Beagle dogs older than 6 months of age were selected. On the day of the assay, the animals were given the corresponding compound by single injection in the cephalic or saphenous vein in a volume of 1 mL/kg for the intravenous group; and by single gavage in a volume of 5 mL/kg for the oral group. The animals were weighed before administration and the dosing volume of the compound was calculated based on their body weight. About 0.5 mL of whole blood was collected from the cephalic or saphenous vein at each time point to prepare plasma for determination of concentrations by high performance liquid chromatography-tandem mass spectrometry (LC-MS/MS). Plasma concentrations were processed using non-compartment model of WinNonlin™ Version 6.3 or above (Pharsight, Mountain View, Calif.) pharmacokinetic software, and pharmacokinetic parameters were calculated using the linear log trapezoidal method.

Assay results: The evaluation results of in vivo PK properties in dogs are shown in Table 16.

TABLE 16
The evaluation results of in vivo PK properties

| | | Trifluoroacetate | The crystal form C |
|---|---|---|---|
| | | salt of the compound | of the compound |
| Administration | Parameters | of formula (I) | of formula (I) |
| Injection | Dose (mg/kg) | 1 | / |
| | T_{1/2} (hr) | 9.83 | |
| | Vdss (L/Kg) | 28.9 | |
| | Cl | 34.2 | |
| | (mL/min/Kg) | | |
| | AUC₀₋ₗₐₛₜ | 765 | |
| | (nm · h) | | |
| Oral | Dose (mg/kg) | 1 | 30 |
| | Cₘₐₓ (nM) | 21.1 | 527 |
| | Tₘₐₓ (h) | 4.00 | 9.00 |
| | AUC₀₋ₗₐₛₜ | 318 | 9399 |
| | (nM/hr) | | |
| | F% | 41.6 | 41.0 |

Assay Conclusion:

The compounds of the present disclosure and the crystal form C of the compound of formula (I) have excellent oral AUC and bioavailability, and have good pharmacokinetic properties, as well as a good linear relationship between exposure and dose in dogs.

### Assay Example 10: Pharmacodynamics Assay of Ba/F3-TEL-FLT3-D835Y Cells in Mice with Subcutaneous Allograft Tumor

Assay Purpose:

The efficacy of the assay compounds in the BALB/c nude mouse model of subcutaneous allografts of Ba/F3-TEL-FLT3-D835Y cells was evaluated in vivo.

Assay Procedure:

Ba/F3-TEL-FLT3-D835Y cell line was cultured in 1640 medium supplemented with 10% fetal bovine serum and 1% double antibody at 37° C. and 5% CO₂, and passaged twice a week. When the cell saturation was 80%-90%, the cells were harvested, counted and inoculated.

When the cells in the logarithmic growth phase reached the required number for the assay, the cells were harvested, and centrifuged at 1000 rpm for 5 min. The supernatant was removed. The cells were resuspended in culture medium, and counted using a cell counter. According to the count results, the original solution was diluted to a cell suspension with a live cell concentration of 1×10⁷ cells/ml with a cell viability of 91.02%, P15 generation. The diluted cell suspension was diluted with matrigel at a ratio of 1:1. After mixing well, the suspension was placed on ice and aspirated with a 1 ml sterile syringe. 0.2 ml of cell suspension was inoculated subcutaneously in the right armpit of each mouse. That is, each mouse was inoculated with 1×10⁶ Ba/F3-TEL-FLT3-D835Y cells. After inoculation, the tumor growth was observed day by day. The mice were randomly grouped according to tumor volume when the average tumor volume reached about 175.77 mm³. Mice were dosed according to their body weight (10 μL/g).

After the start of dosing, the body weight and tumor size of the mice were measured twice a week. Tumor volume was calculated according to the formula: Tumor volume (mm³)=½×(a×b²) (where a represents the long diameter and b represents the short diameter).

Assay results: The tumor inhibitory effects of the compounds are shown in Table 17.

TABLE 17
Results of Ba/F3-TEL-FLT3-D835Y allograft tumor assay

| | | Assay compound | | | | |
|---|---|---|---|---|---|---|
| | | | | The crystal | The crystal | The crystal |
| | | | | form C of | form C of | form C of |
| | | | | the compound | the compound | the compound |
| | | Blank | Compound B | of formula (I) | of formula (I) | of formula (I) |
| Dose (mg/kg) | | 0 | 3 | 1 | 3 | 6 |
| Dosing frequency | | Once a day | Once a day | Once a day | Once a day | Once a day |
| Route of | | Oral | Oral | Oral | Oral | Oral |
| administration | | | | | | |
| Average | Day 0 | 175 ± 2 | 176 ± 2 | 176 ± 2 | 177 ± 2 | 177 ± 2 |
| tumor | Day 15 | 2761 ± 139 | 993 ± 79 | 1160 ± 99 | 683 ± 45 | 77 ± 13 |
| volume | | | | | | |
| (mm³) | | | | | | |

| | | | | | |
|---|---|---|---|---|---|
| % Inhibition rate | — | 68 | 62 | 80 | 103 |

Assay Conclusion:

The compounds of the present disclosure have a significant inhibitory effect on the growth of Ba/F3-TEL-FLT3-D835Y allograft tumor. At the same dose (3 mg/kg), the compounds of the present disclosure exhibit better tumor inhibitory effect than that of compound B. A dose of 6 mg/kg can shrink a tumor.

1. A crystal form A of the compound of formula (I), 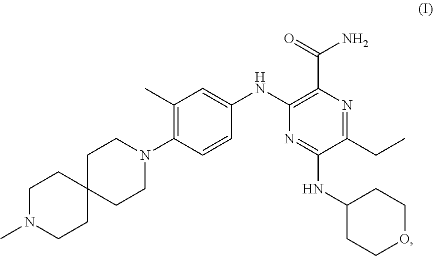 [SMILES: CCc1nc(C(N)=O)c(Nc2ccc(N3CCC4(CCN(C)CC4)CC3)c(C)c2)nc1NC1CCOCC1]
which is characterized by X-ray powder diffraction pattern with characteristic diffraction peaks at 2θ angles of 15.48±0.20°, 19.32±0.20°, and 20.17±0.20°.

2. The crystal form A of the compound of formula (I) according to claim 1, which is characterized by X-ray powder diffraction pattern with characteristic diffraction peaks at 2θ angles of 8.26±0.20°, 14.06±0.20°, 14.83±0.20°, 15.48±0.20°, 18.60±0.20°, 19.32±0.20°, 20.17±0.20°, and 24.28±0.20°.

3. The crystal form A of the compound of formula (I) according to claim 2, which is characterized by X-ray powder diffraction pattern with characteristic diffraction peaks at 2θ angles of 8.26±0.20°, 12.36±0.20°, 14.06±0.20°, 14.83±0.20°, 15.48±0.20°, 16.55±0.20°, 17.29±0.20°, 18.60±0.20°, 19.32±0.20°, 20.17±0.20°, 24.28±0.20°, and 25.51±0.20°; or
wherein the crystal form A of the compound of formula (I) has a XRPD pattern shown in FIG. 1.

4. (canceled)

5. The crystal form A of the compound of formula (I) according to claim 1, which has a thermogravimetric analysis curve with a weight loss of up to 2.65% at 150.0±3° C.; or
wherein the crystal form A of the compound of formula (I) has a TGA curve shown in FIG. 2.

6. (canceled)

7. The crystal form A of the compound of formula (I) according to claim 1, which has a differential scanning calorimetry curve with a starting point of the endothermic peak at 237.1±5° C.; or
wherein the crystal form A of the compound of formula (I) has a DSC curve shown in FIG. 3.

8. (canceled)

9. A crystal form B of the compound of formula (I), 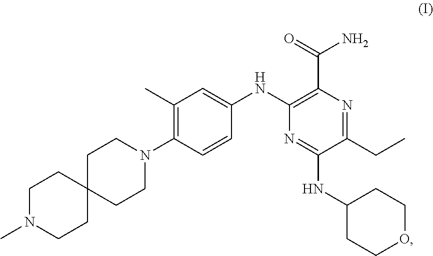 [SMILES: CCc1nc(C(N)=O)c(Nc2ccc(N3CCC4(CCN(C)CC4)CC3)c(C)c2)nc1NC1CCOCC1]
which is characterized by X-ray powder diffraction pattern with characteristic diffraction peaks at 2θ angles of 14.11±0.20°, 19.29±0.20°, and 21.22±0.20°.

10. The crystal form B of the compound of formula (I) according to claim 9, which is characterized by X-ray powder diffraction pattern with characteristic diffraction peaks at 2θ angles of 7.57±0.20°, 14.11±0.20°, 15.16±0.20°, 18.74±0.20°, 19.29±0.20°, 20.68±0.20°, 21.22±0.20°, and 24.28±0.20°.

11. The crystal form B of the compound of formula (I) according to claim 10, which is characterized by X-ray powder diffraction pattern with characteristic diffraction peaks at 2θ angles of 7.05±0.20°, 7.57±0.20°, 14.11±0.20°, 15.16±0.20°, 15.68±0.20°, 17.69±0.20°, 18.74±0.20°, 19.29±0.20°, 20.68±0.20°, 21.22±0.20°, 24.28±0.20°, and 25.17±0.20°; or
wherein the crystal form B of the compound of formula (I) has a XRPD pattern shown in FIG. 5.

12. (canceled)

13. The crystal form B of the compound of formula (I) according to claim 9, which has a thermogravimetric analysis curve with a weight loss of up to 4.20% at 140.0±3° C.; or
wherein the crystal form B of the compound of formula (I) has a TGA curve shown in FIG. 6.

14. (canceled)

15. The crystal form B of the compound of formula (I) according to claim 9, which has a differential scanning calorimetry curve with a starting point of the endothermic peak at 237.2±5° C.; or
wherein the crystal form B of the compound of formula (I) has a DSC curve shown in FIG. 7.

16. (canceled)

17. A crystal form C of the compound of formula (I), 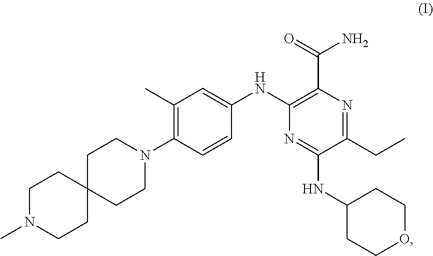 [SMILES: CCc1nc(C(N)=O)c(Nc2ccc(N3CCC4(CCN(C)CC4)CC3)c(C)c2)nc1NC1CCOCC1]
which is characterized by X-ray powder diffraction pattern with characteristic diffraction peaks at 2θ angles of 8.26±0.20°, 19.30±0.20°, and 20.53±0.20°.

18. The crystal form C of the compound of formula (I) according to claim 17, which is characterized by X-ray powder diffraction pattern with characteristic diffraction peaks at 2θ angles of 8.26±0.20°, 12.36±0.20°, 14.07±0.20°, 15.45±0.20°, 18.59±0.20°, 19.30±0.20°, 20.53±0.20°, and 24.29±0.20°.

19. The crystal form C of the compound of formula (I) according to claim 18, which is characterized by X-ray powder diffraction pattern with characteristic diffraction peaks at 2θ angles of 8.26±0.20°, 12.36±0.20°, 14.07±0.20°, 15.45±0.20°, 16.54±0.20°, 17.32±0.20°, 18.59±0.20°, 19.30±0.20°, 20.53±0.20°, 24.29±0.20°, 24.89±0.20°, and 25.49±0.20°; or
wherein the crystal form C of the compound of formula (I) has a XRPD pattern shown in FIG. 8.

20. (canceled)

21. The crystal form C of the compound of formula (I) according to claim 17, which has a thermogravimetric analysis curve with a weight loss of up to 0.71% at 220.0±3° C.; or
wherein the crystal form C of the compound of formula (I) has a TGA curve shown in FIG. 9.

22. (canceled)

23. The crystal form C of the compound of formula (I) according to claim 17, which has a differential scanning calorimetry curve with a starting point of the endothermic peak at 238.1±5° C.; or
wherein the crystal form C of the compound of formula (I), which has a DSC curve as shown in FIG. 10.

24-48. (canceled)

49. A pharmaceutical composition, comprising the crystal form A according to claim 1.

50. (canceled)

51. A pharmaceutical composition, comprising the crystal form B according to claim 9.

52. A pharmaceutical composition, comprising the crystal form C according to claim 17.
